SEQUENCE LISTING (28 sequences)

SEQ ID NO: 1 - DNA, 21 bp, synthetic construct
  source 1..21: mol_type = other DNA
gtgaaaccctgagtcttatcc

SEQ ID NO: 2 - DNA, 20 bp, synthetic construct
  source 1..20: mol_type = other DNA
gaccattctgagacaattcc

SEQ ID NO: 3 - DNA, 23 bp, synthetic construct
  source 1..23: mol_type = other DNA
tgccgactacaagcgaattactg

SEQ ID NO: 4 - DNA, 22 bp, synthetic construct
  source 1..22: mol_type = other DNA
ctgcttgtccagatgacttcgg

SEQ ID NO: 5 - DNA, 24 bp, synthetic construct
  source 1..24: mol_type = other DNA
gcgtggttgccctcttctactttg

SEQ ID NO: 6 - DNA, 23 bp, synthetic construct
  source 1..23: mol_type = other DNA
agtccagtgtccagcccatgatg

SEQ ID NO: 7 - DNA, 20 bp, synthetic construct
  source 1..20: mol_type = other DNA
tccaggatcgaccagggcga

SEQ ID NO: 8 - DNA, 20 bp, synthetic construct
  source 1..20: mol_type = other DNA
aaaagggcgacaacccggcc

SEQ ID NO: 9 - DNA, 19 bp, synthetic construct
  source 1..19: mol_type = other DNA
gtcccgcctcttcactcag

SEQ ID NO: 10 - DNA, 22 bp, synthetic construct
  source 1..22: mol_type = other DNA
gattctggtgtttccccgttgg

SEQ ID NO: 11 - DNA, 20 bp, synthetic construct
  source 1..20: mol_type = other DNA
ctccctctccccgccactcc

SEQ ID NO: 12 - DNA, 21 bp, synthetic construct
  source 1..21: mol_type = other DNA
gggggtgtcttcaatcacgcg

SEQ ID NO: 13 - DNA, 21 bp, synthetic construct
  source 1..21: mol_type = other DNA
aaggtgggctgcgatgagtcc

SEQ ID NO: 14 - DNA, 16 bp, synthetic construct
  source 1..16: mol_type = other DNA
gggtctcctgctggtc

SEQ ID NO: 15 - DNA, 20 bp, synthetic construct
  source 1..20: mol_type = other DNA
gggatgggggaggagcccag

SEQ ID NO: 16 - DNA, 20 bp, synthetic construct
  source 1..20: mol_type = other DNA
aaggtcactgggagggggcg

SEQ ID NO: 17 - DNA, 25 bp, synthetic construct
  source 1..25: mol_type = other DNA
ggactttggattcccgcaggagaag

SEQ ID NO: 18 - DNA, 25 bp, synthetic construct
  source 1..25: mol_type = other DNA
gctgcatcagacagccttgcaggtc

SEQ ID NO: 19 - DNA, 19 bp, synthetic construct
  source 1..19: mol_type = other DNA
gacttcatcttggctgtga

SEQ ID NO: 20 - DNA, 21 bp, synthetic construct
  source 1..21: mol_type = other DNA
tgatttctgctctgacaacct

SEQ ID NO: 21 - DNA, 23 bp, synthetic construct
  source 1..23: mol_type = other DNA
cctggtatgacaatgaatacggc

SEQ ID NO: 22 - DNA, 20 bp, synthetic construct
  source 1..20: mol_type = other DNA
ctccttggaggccatgtagg

SEQ ID NO: 23 - DNA, 19 bp, synthetic construct
  source 1..19: mol_type = other DNA
cgacagtcagccgcatctt

SEQ ID NO: 24 - DNA, 24 bp, synthetic construct
  source 1..24: mol_type = other DNA
ccccatggtgtcttcaatcacgcg

SEQ ID NO: 25 - DNA, 20 bp, synthetic construct
  source 1..20: mol_type = other DNA
aaatctggcaccacaccttc

SEQ ID NO: 26 - DNA, 20 bp, synthetic construct
  source 1..20: mol_type = other DNA
ggggtgttgaaggtctcaaa

SEQ ID NO: 27 - DNA, 21 bp, synthetic construct
  source 1..21: mol_type = other DNA
ttctacaatgacctgggtgtg

SEQ ID NO: 28 - DNA, 20 bp, synthetic construct
  source 1..20: mol_type = other DNA
ggggtgttgaaggtctcaaa

(19) United States
(12) Patent Application Publication
(10) Pub. No.: US 2024/0293430 A1
(43) Pub. Date: Sep. 5, 2024

(54) WITHAFERIN A AND IMMUNE CHECKPOINT BLOCKER COMBINATION THERAPIES

(71) Applicant: University of South Florida, Tampa, FL (US)

(72) Inventors: Subhra Mohapatra, Lutz, FL (US); Shyam MOHAPATRA, Lutz, FL (US); Roukiah Khalil, Tampa, FL (US)

(21) Appl. No.: 18/594,305

(22) Filed: Mar. 4, 2024

Related U.S. Application Data
(60) Provisional application No. 63/505,109, filed on May 31, 2023.
(60) Provisional application No. 63/488,032, filed on Mar. 2, 2023.

(51) Int. Cl. A61K 31/585; A61K 39/395; A61P 35/00

(52) U.S. Cl. CPC A61K 31/585; A61K 39/3955; A61P 35/00

(57) ABSTRACT

The present disclosure provides methods of treating cancer in a subject in need thereof comprising administering to the subject a therapeutically effective amount of withaferin A and an immune checkpoint blocker. Further, the cancer to be treated may be resistant, have developed resistance, or may be susceptible to resistance to treatment with the immune checkpoint blocker alone or in combination with another therapeutic agent other than withaferin A.

### CROSS-REFERENCE TO RELATED APPLICATIONS

This application claims the benefit of priority to United States Provisional Application Nos. 63/488,032, filed Mar. 2, 2023, and 63/505,109, filed May 31, 2023, the disclosures of which are incorporated herein by reference in their entirety.

### STATEMENT REGARDING FEDERALLY SPONSORED RESEARCH OR DEVELOPMENT

This invention was made with government support under Grant No. BX003413 awarded by the Department of Veteran Affairs. The Government has certain rights in the invention.

### INCORPORATION BY REFERENCE

A Sequence Listing conforming to the rules of WIPO Standard ST.26 is hereby incorporated by reference. Said Sequence Listing has been filed as an electronic document via Patent Center encoded as XML in UTF-8 text. The electronic document, created on Mar. 4, 2024, is entitled “11001182US1SEQ.xml”, and is 5,710,798 bytes in size.

### TECHNICAL FIELD

This disclosure relates to methods of treating cancer and, more particularly, to combination therapies for cancers resistant to immune checkpoint blocker therapy.

### BACKGROUND

Lung cancer is the leading cause of cancer deaths, with 127,070 estimated deaths in 2023 in the USA [1]. Despite the abundance of anti-cancer treatments, the five-year survival rate can be as low as 8% in late-stage lung cancer [2]. The role of the immune system in cancer prevention by immunosurveillance was harnessed for the development of immunotherapies, including immune checkpoint blockers (ICBs) [3]. Cancer immunosurveillance is achieved when antigen-presenting cells (APCs) process and present tumor-associated antigens to elicit a cytotoxic T-cell (CTL) response [4]. Consequently, two signals are required for T-cell activation: an antigen-major histocompatibility complex-I (MHC-I) and T-cell receptor (TCR) interaction, and a second-the T-cell co-stimulatory molecule CD-28 and its tumor-expressed cognate ligand B7-1. Following activation, CTLs release apoptotic mediators (e.g., perforin, granzyme B), inducing tumor cell death. To evade immune detection, tumors upregulate the expression of inhibitory immune checkpoint (IC) molecules to counteract T-cell co-stimulatory signals, therefore inactivating CTLs [5]. For example, cytotoxic T-lymphocyte protein 4 (CTLA4) is an inhibitory IC molecule that competitively binds B7-1 and, therefore, inhibits its interaction with CD28. Another IC molecule of significance is the programmed death protein-1 (PD-1), which inhibits T-cell signaling and activation by interacting with its ligand PD-L1 expressed on tumor cells. ICBs are monoclonal antibodies that inhibit the interaction of IC molecules and their ligands, thus restoring T-cell activation. Based on several clinical trials comparing the effectiveness of ICBs to first- or second-line chemotherapies, ICBs were FDA-approved and have been used for lung cancer treatment since 2015 [6]. One advantage of ICBs over other therapies is their ability to activate immunologic memory, leading to a long-lasting response by eliminating recurring tumor cells [7]. However, only 17-20% of treated patients attain a durable response due to tumor-induced immunosuppression [8].

Tumor immunoresistance/immunosuppression can be broadly classified into tumor intrinsic (tumor cell-mediated) or tumor extrinsic resistance (host factors, microbiome, tumor microenvironment (TME)) [9]. Tumor intrinsic resistance involves poor immunogenicity, defective interferon signaling, and the upregulation of inhibitory IC molecules, while tumor-extrinsic resistance includes lack of immune infiltration or immunosuppressive cell infiltration (e.g., regulatory T-cells (T-regs) and myeloid-derived suppressor cells (MDSCs)) [10]. Therefore, certain factors/biomarkers are associated with a better response to ICB therapy [11]. For instance, non-small cell lung cancer (NSCLC) patients having a higher expression of the IC molecule programmed death ligand 1 (PD-L1) show a response to ICBs [12]. Furthermore, patients with higher tumor-infiltrating lymphocytes (immunologically hot) have a better response than tumors that lack immune cell infiltration (immunologically cold) [13]. Other predictors of patient response include microsatellite instability and tumor mutational burden [14]. Therefore, biomarkers such as higher PD-L1 expression levels and tumor immune infiltration are associated with better response and are used to assess whether a patient is a good candidate for ICB therapy.

To overcome the immune resistance and improve patient response to ICBs, current trials attempt to combine ICBs with drugs/radiation that target the immunosuppressive mechanisms in the TME [15]. For example, anthracyclines and oncolytic viruses induce immunogenic cell death (ICD) in which dying tumor cells release or express danger-associated molecular patterns (DAMPs) such as calreticulin (CRT), High Mobility Group Box-1 (HMGB-1), and heat shock proteins (HSP-70 and HSP-90) [16]. Consequently, APCs detect DAMPs as ‘eat-me signals' and engulf dying tumor cells to activate an anti-tumor CTL response. As a result, ICD can convert an immunologically cold into an immunologically hot TME [17]. Furthermore, radiotherapy and some chemotherapeutics (e.g., mitomycin C and cisplatin) that increase PD-L1 expression on tumor cells sensitize lung cancer to α-PD-L1 therapy [15,18,19]. However, many of these combinations are not clinically applicable due to the additive side effects of the individual agents, including cardiotoxicity [20,21]. Therefore, there is still a clear need for therapies that expand the use of ICBs to a wider patient population by overcoming immune resistance without additive toxicity.

### SUMMARY

The present disclosure provides methods of treating a cancer in a subject in need thereof. In some aspects, the method comprises administering to the subject a therapeutically effective amount of withaferin A. In some aspects, the method comprises administering to the subject a therapeutically effective amount of an immune checkpoint inhibitor. In some aspects, withaferin A and the immune checkpoint inhibitor are administered in combination or alternation to the subject.

In some aspects, the cancer is resistant or has developed resistance to treatment with the immune checkpoint blocker alone or with the immune checkpoint blocker in combination with another therapeutic agent other than withaferin A. In some aspects, the cancer is susceptible to developing resistance to treatment with the immune checkpoint blocker alone or with the immune checkpoint blocker in combination with another therapeutic agent other than withaferin A.

In some aspects, the cancer is selected from basal cell carcinoma, bladder cancer, breast cancer, cervical cancer, cholangiocarcinoma, colorectal cancer, endometrial cancer, esophageal cancer, head and neck cancer, hepatocellular carcinoma, Hodgkin lymphoma, melanoma, mesothelioma, Merkel cell carcinoma, non-small cell lung cancer, renal cell carcinoma, small cell lung cancer, squamous cell carcinoma, stomach cancer, and urothelial carcinoma. In some aspects, the cancer is selected from breast cancer, colorectal cancer, and non-small cell lung cancer.

In some aspects, the immune checkpoint blocker comprises an inhibitor of CTLA-4.

In some aspects, the immune checkpoint blocker comprises an inhibitor of CTLA-4 selected from ipilimumab and tremelimumab.

In some aspects, the immune checkpoint blocker comprises an inhibitor of PD-1. In some aspects, the immune checkpoint blocker comprises an inhibitor of PD-1 selected from pembrolizumab, nivolumab, cemiplimab, dostarlimab, retafanlimab, toripalimab, vopratelimab (JTX-4014), spartalizumab (PDR001), cambrelizumab (SHR1210), sintilimab (I1BI308), tislelizumab (BGB-A317), INCMGA00012 (MGA012), AMP-224, AMP-514 (MEDI0680), and acrixolimab (YBK-006). In some aspects, the immune checkpoint blocker comprises an inhibitor of PD-1 selected from nivolumab, pembrolizumab, cemiplimab, and dostarlimab.

In some aspects, the immune checkpoint blocker comprises an inhibitor of PD-L1. In some aspects, the immune checkpoint blocker comprises an inhibitor of PD-L1 selected from atezolizumab, avelumab, durvalumab, KN035, cosibelimab (CK-301), AUNP12, CA-170, and BMS-986189. In some aspects, the immune checkpoint blocker comprises an inhibitor of PD-L1 selected from atezolizumab, avelumab, and durvalumab.

In some aspects, the method further comprises administering one or more additional therapeutic agents. In some aspects, the method further comprises administering a therapeutically effective amount of ionizing radiation.

In some aspects, the subject is a human.

The details of one or more aspects of the disclosure are set forth in the accompanying drawings and the description below. Other features, objects, and advantages of the disclosure will be apparent from the description, the drawings, and the claims.

### DESCRIPTION OF DRAWINGS

FIGS. 1A-lE depict that WFA induces ER stress-mediated apoptosis in NSCLC cell lines. (FIG. 1A) NSCLC cells were plated on 96-well plates and treated with different concentrations of WFA (100.1 uM) in triplicates. Forty-eight hours post-treatment, cell viability was determined using Cell Titer-Glo assay. Luminescence values were normalized to control untreated cells and IC50 values are represented as the mean of two independent experiments ±SEM. (FIG. 1B) NSCLC cells were treated WFA for forty-eight hours, then collected to measure apoptotic cell death using Annexin V assay in LLC cells, H1650 cells, and A549 cells. Percentages of early (annexin*DAPI-) and late (annexin*DAPI+) apoptotic cells are represented as means of two separate experiments ±SEM and statistical significance was calculated using one-way ANOVA and a Fisher LSD post hoc test was used to compare the mean of each group to that of the control group. (FIGS. 1C-1D) WFA treated cells were collected in Trizol and total RNA was isolated to measure the levels of Bax (FIG. 1C) and Bcl2 (FIG. 1D) mRNA using qRT-PCR. PCR experiments were repeated at least twice, and one representative experiment is shown as mean±SEM of the technical replicates. (FIG. 1E) Western blot of ER stress markers p-eIF-2 and CHOP was performed using total lysates of WFA-treated LLC cells. The quantification of western blot band density was performed using Image J software v.1.54d and normalized to housekeeping control protein in LLC cells (0-actin). The mean fold change in band density from the control is shown in the bar graphs ±SEM and one-way ANOVA and a Fisher LSD post hoc test were used to calculate statistical significance where * denotes p<0.05, ** p<0.01, *** p<0.001, **** p<0.0001. Data were obtained from two independent experiments.

FIGS. 2A-2E depict that WFA induces ICD in NSCLC cell lines. Cells were treated with WFA for 48 h then collected using Accutase cell detachment solution. (FIGS. 2A-2C) CRT levels were determined by flow cytometry in LLC (FIG. 2A), H1650 (FIG. 2B), and A549 (FIG. 2C) cells. (FIG. 2D) The levels of secreted HMGB-1 in LLC cell treated with 0.6 μM WFA or 30 nM doxorubicin were measured by ELISA and the means of two independent experiments ±SEM are represented in the bar graph. (FIG. 2E) Bone marrow-derived DC were co-cultured with WFA-pretreated LLC cells (24 h) in a ratio 5:1 of DC:LLC cells for 24 h. DC activation markers (CD80, CD86, and MHC-II) were examined by flow cytometry. The means of at least 2 independent experiments ±SEM is represented, and one-way ANOVA was used to calculate statistical significance where * denotes p<0.05, ** denote p<0.01, *** denote p<0.001, and **** denote p<0.0001.

FIGS. 3A-3F depict that WFA upregulates PD-L1 surface expression in NSCLC cell lines. Cells were treated with WFA for 48 h. (FIGS. 3A, 3C, 3E) PD-L1 surface expression determined using flow cytometry LLC (FIG. 3A), H1650 (FIG. 3C), and A549 (FIG. 3E). (FIGS. 3B, 3D, 3F) PD-L1 expression was measured using qRT-PCR LLC (FIG. 3B), H1650 (FIG. 3D), A549 (FIG. 3F). PCR experiments were repeated twice and the mean±SEM of the technical replicates are represented. For the rest, the means of two independent experiments ±SEM are shown, and statistical significance was calculated using one-way ANOVA and a Fisher LSD post hoc test where * is p<0.05, ** is p<0.01, and **** is p <0.0001.

FIGS. 4A-4I depict that WFA-mediated ROS production is essential for PD-L1 upregulation and can be abrogated using NAC. NSCLC cell lines were treated with WFA (0.6 μM in LLC, H1650 cells, 4 μM in A549 cells), or a combination of WFA+5 mM NAC for 24 h. (FIGS. 4A, 4D, 4G) The change in ROS levels were measured in LLC (FIG. 4A), H1650 (FIG. 4D), and A549 (FIG. G). (FIGS. 4B, 4E, 4H) by flow cytometry. The change in PD-L1 levels was measured in LLC (FIG. 4B), H1650 (FIG. 4E), and A549 (FIG. 4H) cell lines using flow cytometry and qRT-PCR. (FIG. 4C, 4F, 4I) Ecto-CRT levels were measured in LLC (FIG. 4C), H1650 (FIG. 4F), and A549 (FIG. 41) by flow cytometry. Means of two independent experiments ±SEM are shown, except for the PCR experiments that were repeated twice and for which one representative experiment is shown. Statistical significance was calculated using one-way ANOVA and a Fisher LSD post hoc test where * is p<0.05, ** is p<0.01,*** is p<0.001, and **** is p<0.0001.

FIGS. 5A-5G depict that WFA sensitizes flank tumors to α-PD-L1 therapy and targets immunosuppressive MDSCs and T-regs. (FIG. 5A) Establishment of LLC flank tumors and treatment schedule. (FIG. 5B) Tumor volume measurement in mm³ and tumor weight at the endpoint of the experiment. (FIG. 5C) Change in the levels of ecto-CRT in tumors was measured using flow cytometry. (FIGS. 5D-5G) Flow cytometry was used to determine the change in immune cell infiltration in collected tumors. WFA treatment increased CD3 (FIG. 5D) and CD8 T-cell (FIG. 5E) infiltration and activation markers 4-1BB and CD69. Moreover, WFA reduced the immunosuppressive T regs (FIG. 5F) and CD11b+Gr1+MDSC (FIG. 5G) populations. The means of two independent experiments ±SEM are shown, and the statistical significance was calculated using one-way ANOVA and a Fisher LSD post hoc test where * and #represent p<0.05 and ** and ##represent p<0.01. * Symbols refer to the difference between the vehicle control and WFA, while #refers to comparisons between the vehicle control and WFA+α-PD-L1 combination.

FIGS. 6A-6E depict that ROS partially contributes to WFA-mediated anti-cancer effectiveness and immunomodulation in vivo. (FIG. 5A) Establishment of LLC flank tumors and treatment schedule. (FIG. 6B) Tumor volume measurement in mm³ and tumor weight at the endpoint of the experiment. (FIGS. 6C-6E) Flow cytometry was used to determine the change in immune cell infiltration in collected tumors. NAC treatment reversed WFA mediated increase in CD8 T-cell (FIG. 6C) infiltration while increasing the immunosuppressive CD11b+Gr1+MDSCs (FIG. 6D) and CD25+CD4+T-reg (FIG. 6E) populations. Statistical significance was calculated using one-way ANOVA and each group was compared to the vehicle control using the Fisher LSD post hoc test where #represents p <0.05 and ##represents ap<0.01.

FIGS. 7A-7I depict qRT-PCR analysis of the change in expression of pro-apoptotic and anti-apoptotic induced by WFA treatment. WFA increased the mRNA levels of proapoptotic protein Bak-1 in (FIG. 7A) LLC cells and BAD in (FIG. 7C) H1650 cells. WFA also decreased the mRNA levels of the anti-apoptotic protein Survivin and Xiap in both (FIG. 7B) LLC and (FIG. 7D) A549cell line. PCR was repeated twice, and one representative experiment is shown as mean±SEM of 3 technical replicates. Western blotting shows that WFA treatment increased PARP cleavage in (FIG. 7E) LLC and H1650 cells and decreases the anti-apoptotic protein BCL-XL in (FIG. 7F) H1650 cells. (FIGS. 7G-7I) Western blots.

One-way ANOVA and Fisher LSD post hoc test were used to compare the treatment groups to the control. * p<0.05, **p<0.01, ***p<0.001.

FIGS. 8A-8G depict that WFA induced ICD and PD-L1 upregulation in colorectal cancer cell lines. WFA increased the levels of ecto-CRT in the murine colorectal cancer cell line (FIG. 8A) MC-38 and human cell line (FIG. 8B) HCT-116. WFA increased the expression of PD-L1 in both (FIG. 8C) MC-38 and (FIG. 8D) HCT-116 cells. (FIG. 8E) Western blotting shows that WFA treatment increases the levels of PD-L1 protein in LLC, H1650 and A549 NSCLC cell lines. H1650 cells (at a higher density) were treated with a higher concentration of WFA (2 and 4 μM) to facilitate cell collection and protein isolation. The band density was quantified using Image J software and normalized to the housekeeping protein. The fold change from untreated control was averaged from two independent experiments ±SEM and one-way ANOVA was used to calculate the statistical significance. A Fisher LSD post hoc test was performed to compare the treatments to the control in (FIG. 8E) LLC, (FIG. 8F) H1650, (FIG. 8G) A549 cells. * p<0.05, ** p<0.01, *** p<0.001, **** p<0.0001.

FIG. 9 depicts the gating strategy used for the DC ex-vivo activation assay. Hierarchical gating was used to identify the total cell population followed by single live cells the CD11C+DC. Out of the CD11C population, the expression of DC cell activation markers was determined based on the fluorescence minus one (FMO) controls.

FIGS. 10A-10I depict that WFA treatment increased the mRNA levels of IFNα in (FIG. 10A) LLC and (FIG. 10B) H1650 cell lines. ROS is the main regulator of WFA-induced PD-L1 upregulation. Flow cytometry analysis shows thatNAC (5 mM) or GSH (5 mM) abrogated WFA-mediated PD-L1 upregulation in (FIG. 10C) LLC, (FIG. 10D) H1650 and (FIG. 10E) A549 (FIG. 10F) MC-38, (FIG. 10G) HCT-116 colorectal cancer cell lines, (FIG. 10H) 4T1, and (FIG. 101) MDA-MB-231 breast cancer cell lines. All cells were treated with 0.6 μM WFA except A549, 4t1 and MDA-MB-231 cells which were treated with 4 μM, 1 μM, 2 μM WFA, respectively. (FIG. 10F) qRT-PCR shows that NAC abrogates WFA-mediated PD-L1 upregulation in MC-38 cells. PCR experiments were repeated twice, and one representative experiment is shown as mean±SEM of 3 technical replicates. Flow cytometry experiments were performed twice and the mean±SEM of two representative experiments is shown. Treatments were compared to the control using one-way ANOVA and Fisher LSD post hoc test. * p<0.05, ** p<0.01, *** p<0.001, **** p<0.0001.

FIG. 11 depicts that a gene interaction network between the PDL1 signaling pathway and reactive oxygen species (nitric oxide, oxygen radical, hydrogen peroxide, and lipoxygenase) signaling was generated using known connections obtained from the Ingenuity Pathway Analysis database. Fold change data from MCF-7 breast cancer cells treated with withaferin A (700 nM for 72 hr) was obtained from NCBI Gene Omnibus Expression Database (Series GSE53049) and overlaid onto the network to yield predictions of activation/inhibition in the IPA software.

FIGS. 12A-12D depicts the gating strategy used to determine tumor immune infiltration in LLC tumors and spleens. Hierarchical gating was used to identify the total cell population followed by single live cells then (FIG. 12A) the CD11b+myeloid cells. Out of the CD11b population, we gated the monocytic CD11b+Ly6C+ and CD11b+Ly6G+granulocytic MDSCs. (FIG. 12B) For the T-cell panel, the total CD-3 population was gated out of the live single cells. Consequently, CD4, CD25+CD4+Tregs and CD8 population were gated out of CD-3 cells based on the FMO controls. (FIGS. 12C-12D) Mice body weights were measured at the endpoint of the experiment and represented as mean±SEM. The means were compared to the vehicle control using Fisher LSD post-hoc test. * p<0.05.

FIGS. 13A-13I depict investigating the downstream molecular regulators that may be involved in WFA-mediated PD-L1 upregulation. LLC (FIG. 13A) and H1650 (FIG. 13B) were treated with either WFA or WFA+STATTIC for twenty-four hours then PD-L1 expression was measured using flow cytometry. (FIG. 13C) LLC cells were treated with WFA or WFA+PX-478 then collected for PD-L1 measurement using flow cytometry. LLC (FIG. 13D), H1650 (FIG. 13E), and A540 cells (FIG. 13F) were treated with WFA or WFA+Brusatol then collected for PD-L1 measurement using flow cytometry. LLC (FIG. 13G) or H1650 (FIG. 13H) were transfected with a NRF-2 or scramble siRNA then treated with WFA for twenty-four hours to measure PD-L1 levels by flow cytometry. (FIG. 131)NRF-2 knockdown was confirmed by western blotting of scramble and siRNA-transfected cells. Flow cytometry experiments were repeated at least twice and the means±SEM are shown in the figures and compared using one-way ANOVA and Fisher LSD post hoc test. * p<0.05, **p<0.01, ***p<0.001, ****p<0.0001.

Like reference symbols in the various drawings indicate like elements.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, benefiting from the teachings presented in the descriptions herein and the associated drawings. Therefore, it is understood that the disclosures are not limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Nothing herein is to be construed as an admission that the present invention is not entitled to antedate such publication by prior invention. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

### Definitions

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of” Similarly, “consisting essentially of” is intended to include examples encompassed by the term “consisting of.”

As used in the specification and the appended claims, the singular forms “a,” “an,” and “the” include plural referents unless the context dictates otherwise.

Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’‘about y,’ and ‘about z’ as well as the ranges of ‘less than x,’ ‘less than y.’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’In addition, the phrase “about ‘x’ to ‘y’,” where ‘x’ and ‘y’ are numerical values, includes “about ‘x’ to about ‘y’.” Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated+10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, the term “therapeutically effective amount” refers to an amount sufficient to achieve the desired therapeutic result or to have an effect on undesired symptoms but generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the particular compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to permanently halt the progression of the disease. This can be monitored by routine diagnostic methods known to one of ordinary skill in the art for any particular disease. The desired response to treatment of the disease or condition can also be delaying the onset or even preventing the onset.

For example, it is well within the skill of the art to start doses of a compound at levels lower than those required to achieve the desired therapeutic effect and to increase the dosage gradually until the desired effect is achieved. If desired, the effective daily dose can be divided into multiple doses for administration. Consequently, single-dose compositions can contain such amounts or submultiples thereof to make up the daily dose. The individual physician can adjust the dosage in the event of any contraindications. It is generally preferred that a maximum dose of the pharmacological agents of the invention (alone or in combination with other therapeutic agents) be used, that is, the highest safe dose according to sound medical judgment. However, a patient may insist on a lower or tolerable dose for medical reasons, psychological reasons, or virtually any other reason.

A response to a therapeutically effective dose of a disclosed compound or composition can be measured by determining the physiological effects of the treatment or medication, such as the decrease or lack of disease symptoms following the administration of the treatment or pharmacological agent. Other assays will be known to one of ordinary skill in the art and can be employed for measuring the level of the response. The amount of a treatment may be varied, for example, by increasing or decreasing the amount of a disclosed compound or pharmaceutical composition, changing the disclosed compound or pharmaceutical composition administered, changing the route of administration, changing the dosage timing, and so on. Dosage can vary and can be administered in one or more dose administrations daily for one or several days. Guidance can be found in the literature for appropriate dosages for given classes of pharmaceutical products.

As used herein, “optional” or “optionally” means that the subsequently described event or circumstance can or cannot occur. The description includes instances where said event or circumstance occurs and those where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to a human and constituents thereof.

As used herein, “treating” and “treatment” generally refer to obtaining a desired pharmacological or physiological effect. The effect can be but does not necessarily have to be prophylactic in preventing or partially preventing a disease, symptom, or condition such as a cancer. The effect can be therapeutic regarding a partial or complete cure of a disease, condition, symptom, or adverse effect attributed to the disease, disorder, or condition. The term “treatment” as used herein can include any treatment of a disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to both therapeutic treatment alone, prophylactic treatment alone, or both therapeutic and prophylactic treatment. Those in need of treatment (i.e., subjects in need thereof) can include those already with the disorder or those in which the disorder is to be prevented. As used herein, the term “treating” can include inhibiting the disease, disorder, or condition, e.g., impeding its progress, and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound or a pharmaceutical composition thereof calculated to produce the desired response or responses in association with its administration.

As used herein, “therapeutic” can refer to treating, healing, or ameliorating a disease, disorder, condition, or side effect or decreasing the rate of advancement of a disease, disorder, condition, or side effect.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation. Although the operations of exemplary aspects of the disclosed method may be described in a particular sequential order for convenient presentation, it should be understood that disclosed aspects can encompass an order of operations other than the particular sequential order disclosed. For example, operations described sequentially may, in some cases, be rearranged or performed concurrently. Further, descriptions and disclosures provided in association with one particular aspect are not limited to that aspect and may be applied to any aspect disclosed.

As used herein, the term “substantially” means that the subsequently described event or circumstance completely occurs or that the subsequently described event or circumstance generally, typically, or approximately occurs.

Still further, the term “substantially” can, in some aspects, refer to at least about 90 %, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

As used herein, the term “substantially,” in, for example, the context “substantially identical” or “substantially similar,” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates, and other isomers, such as rotamers, as if each is specifically described unless otherwise indicated or otherwise excluded by context. It is to be understood that the compounds provided herein may contain chiral centers. Such chiral centers may be of either the (R-) or (S-) configuration. The compounds provided herein may either be enantiomerically pure or diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like) or by reacting free base forms of these compounds with a stoichiometric amount of the appropriate acid. Such reactions are typically carried out in water, in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts that are acceptable for human consumption and the quaternary ammonium salts of the parent compound formed, for example, from inorganic or organic salts. Examples of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Suitable counterions found in pharmaceutically acceptable salts described herein include but are not limited to, cations such as calcium, chloroprocaine, choline, diethanolamine, ethanolamine, ethylenediamine, meglumine, potassium, procaine, sodium, triethylamine, and zinc, and anions such as acetate, aspartate, benzenesulfonate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, succinate, sulfate, tartrate, teoclate, and tosylate. Lists of additional suitable salts may be found, e.g., in Remington's Pharmaceutical Sciences, 17^{th} ed., Mack Publishing Company, Easton, PA., p. 1418 (1985).

As used herein, the term “derivative” refers to a compound having a structure derived from the structure of a parent compound (e.g., a compound disclosed herein) and whose structure is sufficiently similar to those disclosed herein and based upon that similarity would be expected by one skilled in the art to exhibit the same or similar activities and utilities as the claimed compounds, or to induce, as a precursor, the same or similar activities and utilities as the claimed compound. Exemplary derivatives include but are not limited to, salts, esters, amides, salts of esters or amides, and N-oxides of a parent compound.

The present disclosure also includes compounds with at least one desired isotopic substitution of an atom at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, 17O 180, ¹⁸F, ³¹P³²p, ³⁵S, ³⁶Cl, and ¹²⁵I, respectively. In one aspect, isotopically labeled compounds can be used in metabolic studies (with ¹⁴C), reaction kinetic studies (with, for example, ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F-labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example, deuterium (²H) and tritium (³H), may optionally be used anywhere in described structures that achieve the desired result. Alternatively, or in addition, isotopes of carbon, e.g., ¹³C and ¹⁴C, may be used. In one aspect, the isotopic substitution is replacing hydrogen with deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example, deuterium substitutions, can be partial or complete. Partial deuterium substitution means that at least one hydrogen is substituted with deuterium. In certain aspects, the isotope is 80, 85, 90, 95, or 99% or more enriched in an isotope at any location of interest. In some aspects, deuterium is 80, 85, 90, 95, or 99% enriched at a desired location. Unless otherwise stated, the enrichment at any point is above natural abundance and, in an aspect, is enough to alter a detectable property of the compounds as a drug in a human.

The compounds of the present disclosure may form a solvate with solvents (including water). Therefore, in one aspect, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone, and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D₂0, d6-acetone, or d₆-DMSO. A solvate can be in a liquid or solid form.

A “prodrug,” as used herein, means a compound which, when administered to a host in vivo, is converted into a parent drug. As used herein, the term “parent drug” means any of the presently described compounds herein. Prodrugs can be used to achieve any desired effect, including to enhance the properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain aspects, the prodrug renders the parent compound more lipophilic. In certain aspects, a prodrug can be provided that has several prodrug moieties in a linear, branched, or cyclic manner. For example, non-limiting aspects include the use of a divalent linker moiety such as a dicarboxylic acid, amino acid, diamine, hydroxycarboxylic acid, hydroxyamine, di-hydroxy compound, or other compounds that have at least two functional groups that can link the parent compound with another prodrug moiety and are typically biodegradable in vivo. In some aspects, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂-S-(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some aspects, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched, or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Methods of Treatment

The present disclosure provides methods for treating a cancer in a subject in need thereof. In some aspects, the method comprises administering to the subject a therapeutically effective amount of withaferin A. In some aspects, the method further comprises administering to the subject a therapeutically effective amount of an immune checkpoint blocker. In some aspects, withaferin A is administered in combination or alternation with the immune checkpoint blocker. In some aspects, the cancer is resistant to treatment with the immune checkpoint blocker alone or in combination with another therapeutic agent other than withaferin A. In some aspects, the cancer has developed resistance to treatment with the immune checkpoint blocker alone or in combination with another therapeutic agent other than withaferin A. In some aspects, the cancer is susceptible to developing resistance to treatment with an immune checkpoint blocker alone or in combination with another therapeutic agent other than withaferin A.

In some aspects, the methods can further comprise administering one or more additional therapeutic agents, for example, anti-cancer agents or anti-inflammatory agents. The methods can further comprise administering a therapeutically effective amount of ionizing radiation to the subject.

The disclosed methods can optionally include identifying a patient who is or can be in need of treatment of an oncological disorder. The patient can be a human or other mammals, such as a primate (monkey, chimpanzee, ape, etc.), dog, cat, cow, pig, or horse, or other animals having an oncological disorder. In some aspects, the subject can receive the therapeutic compositions prior to, during, or after surgical intervention to remove part or all of a tumor.

In some aspects, an assay can be used to determine whether the subject has resistance to treatment with the immune checkpoint blocker alone using a sample (e.g., a biological sample or a biopsy sample (e.g., a paraffin-embedded biopsy sample) from a subject. Representative examples of such assays can include, for example, next-generation sequencing, immunohistochemistry, fluorescence microscopy, break-apart FISH analysis, Southern blotting, Western blotting, FACS analysis, Northern blotting, and PCR-based amplification (e.g., RT-PCR). As is well-known in the art, the assays are typically performed, e.g., with at least one labeled nucleic acid probe or at least one labeled antibody or antigen-binding fragment thereof. Assays can utilize other detection methods known in the art for detecting resistance to immune checkpoint blocker therapy.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue, and most invade surrounding tissues, can metastasize to several sites, are likely to recur after attempted removal, and may cause the death of the patient unless adequately treated. As used herein, the term neoplasia is used to describe all cancerous disease states and embraces or encompasses the pathological process associated with malignant, hematogenous, ascitic, and solid tumors. The cancers that may be treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas that may be treated by the compositions of the present disclosure include but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma(MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas that may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenstram macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example, germinomatous (such as germinoma, dysgerminoma, and seminoma), non-germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example, hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndromes, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AJDs-related cancers such as Kaposi sarcoma; epithelioid hemangioendothelioma; desmoplastic small round cell tumor; and liposarcoma.

In some aspects, the cancer is selected from basal cell carcinoma, bladder cancer, breast cancer, cervical cancer, cholangiocarcinoma, colorectal cancer, endometrial cancer, esophageal cancer, head and neck cancer, hepatocellular carcinoma, Hodgkin lymphoma, melanoma, mesothelioma, Merkel cell carcinoma, non-small cell lung cancer, renal cell carcinoma, small cell lung cancer, squamous cell carcinoma, stomach cancer, and urothelial carcinoma.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors, including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some aspects, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors, including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity and in vivo activity in animal models. Methods for the extrapolation of effective dosages in mice and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex, and extent of the disease in the patient and can be determined by one of skill in the art. The dosage can be adjusted by the individual physician in the event of any counterindications. Dosage can vary and can be administered in one or more dose administrations daily, for one or several days.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can also be systemically administered, such as intravenously or orally, optionally in combination with a pharmaceutically acceptable carrier, such as an inert diluent or an assimilable edible carrier for oral delivery. In addition, the active compound can be incorporated into sustained-release preparations and/or devices.

For the treatment of oncological disorder, compounds, agents, and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds, agents, and compositions disclosed herein can be used in methods of treating cancer wherein the patient is to be treated or is or has been treated with mitotic inhibitors such as taxol or vinblastine, alkylating agents such as cyclophosphamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, imatinid or trastuzumab. These other substances or radiation treatments can be given at the same time as or at different times from the compounds disclosed herein. Examples of other suitable chemotherapeutic agents include but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uraxil, temozolomide, thiotepa, tioguanine/thioguanine, topotexan, treosulfan, vinblastine, vincristine, vindesine, and vinorelbine. Examples of suitable immunotherapeutic agents include but are not limited to, alemtuzumab, cetuximab, gemtuzumab, iodine 131 tositumomab, rituximab, and trastuzumab. Cytotoxic agents include, for example, radioactive isotopes and toxins of bacterial, fungal, plant, or animal origin. Also disclosed are methods of treating an oncological disorder comprising administering an effective amount of a compound described herein prior to, subsequent to, and/or in combination with administration of a chemotherapeutic agent, an immunotherapeutic agent, a radiotherapeutic agent, or radiotherapy.

### Withaferin A

In some aspects, the methods described herein comprise administering to a subject a therapeutically effective amount of Withaferin A (WFA). Withaferin A has the following chemical structure:

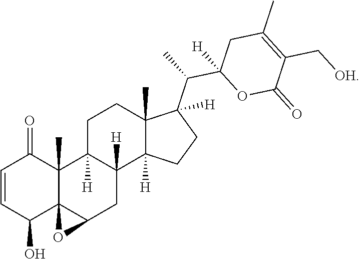

Withaferin A (WFA) is a broad-spectrum anticancer steroidal lactone isolated from the leaves and roots of Withania Somnifera (WS) [22]. Mechanistically, WFA induces apoptotic and ferroptotic cell death, and inhibits angiogenesis, metastasis, and cancer stemness [23-25]. Additionally, WFA exerts its anti-cancer effects by targeting several downstream molecules. For instance, WFA was found to activate Liver-x-receptor signaling and inhibit NF-1B, VEGF, MMPs, cyclins, CDKs, and EGFR [26-29]. One important mechanism of WFA-induced cytotoxicity in cancer cells is mitochondrial dysfunction and subsequent increased oxidative stress and reactive oxygen species (ROS) production [30].

Specifically, WFA was shown to inhibit complex III function and disturb the electron transport chain, leading to increased ROS production [31]. Although WFA is toxic to cancer cells, studies show that it can protect normal non-cancerous tissues, including lung fibroblasts, neurons, and cardiomyocytes, from injury. For instance, WFA was found to protect cardiomyocyte cells from oxidative damage by activating several antioxidant pathways, including SOD-2 and SOD-3 [32]. Moreover, normal cells such as WI-38 lung fibroblasts and PBMCs remain viable at WFA concentrations that were toxic to A549 NSCLC cells [33]. This indicates that WFA can play a dual role as a pro-oxidant or an antioxidant agent based on the cellular context. Although WS was found to be an immunostimulant, only a few studies show the effect of WFA on anti-tumor immunity. In BALB/c mice, WS increases B- and T-cell proliferation as well as T-helper 1 response [34]. Moreover, a clinical study shows that WS extract increases TBNK and IgG levels in healthy subjects [35]. In a breast cancer immunocompetent mouse model, WFA was shown to target the immunosuppressive MDSC population by reducing their tumor infiltration and immunosuppressive function [36].

### Immune Checkpoint Blockers

In some aspects, the methods described herein comprise administering to a subject a therapeutically effective amount of an immune checkpoint blocker. Immune checkpoint blockers are a form of cancer immunotherapy that targets immune checkpoints, key regulators of the immune system that, when stimulated, can dampen the immune response to an immunologic stimulus. Some cancers can protect themselves from attach by stimulating immune checkpoint targets. Immune checkpoint blockers can block inhibitory checkpoints, restoring immune system function. Currently approved immune checkpoint blockers target the molecules CTLA-4, PD-1, and PD-L1. PD-1 is the transmembrane programmed cell death 1 protein (also called PDCD1 and CD279) that interacts with PD-L1 (LD-1 ligand 1, or CD274). PD-L1 on the cell surface binds to PD-1 on an immune cell surface, which inhibits immune cell activity. Among PD-L1 functions are a key regulatory role of T cell activity. Cancer-mediated upregulation of PD-L1 on the cell surface may inhibit T cells that might otherwise attack. Antibodies that bind to either PD-1 or PD-L1 and, therefore, block the interaction may allow the T-cells to attack the tumor.

In some aspects, the immune checkpoint blocker comprises an inhibitor of cytotoxic T-lymphocyte associated protein 4 (CTLA-4). In some aspects, the immune checkpoint blocker comprises ipilimumab. In other aspects, the immune checkpoint blocker comprises tremelimumab.

In some aspects, the immune checkpoint blocker comprises an inhibitor of PD-1. In some aspects, the immune checkpoint blocker comprises pembrolizumab. In some aspects, the immune checkpoint blocker comprises nivolumab. In some aspects, the immune checkpoint blocker comprises cemiplimab. In some aspects, the immune checkpoint blocker comprises dostarlimab. In some aspects, the immune checkpoint blocker comprises retafanlimab. In some aspects, the immune checkpoint blocker comprises toripalimab. In some aspects, the immune checkpoint blocker comprises vopratelimab (JTX-4014). In some aspects, the immune checkpoint blocker comprises spartalizumab (PDR001). In some aspects, the immune checkpoint blocker comprises cambrelizumab (SHR1210). In some aspects, the immune checkpoint blocker comprises sintilimab (IBI308). In some aspects, the immune checkpoint blocker comprises tislelizumab (BGB-A317). In some aspects, the immune checkpoint blocker comprises INCMGA00012 (MGA012). In some aspects, the immune checkpoint blocker comprises AMP-224. In some aspects, the immune checkpoint blocker comprises AMP-514 (MEDI0680). In some aspects, the immune checkpoint blocker comprises acrixolimab (YBK-006).

In some aspects, the immune checkpoint blocker comprises an inhibitor of PD-L1. In some aspects, the immune checkpoint blocker comprises atezolizumab. In some aspects, the immune checkpoint blocker comprises avelumab. In some aspects, the immune checkpoint blocker comprises durvalumab. In some aspects, the immune checkpoint blocker comprises KN035. In some aspects, the immune checkpoint blocker comprises cosibelimab (CK-301). In some aspects, the immune checkpoint blocker comprises AUNP12. In some aspects, the immune checkpoint blocker comprises CA-170. In some aspects, the immune checkpoint blocker comprises BMS-986189.

### Pharmaceutical Compositions

The compounds as used in the methods described herein can be administered by any suitable method and technique presently or prospectively known to those skilled in the art. For example, the active components described herein can be formulated in a physiologically—or pharmaceutically-acceptable form and administered by any suitable route known in the art, including, for example, oral and parenteral routes of administration. As used herein, the term “parenteral” includes subcutaneous, intradermal, intravenous, intramuscular, intraperitoneal, and intrasternal administration, such as by injection. Administration of the active components of their compositions can be a single administration or at continuous and distinct intervals as can be readily determined by a person skilled in the art.

Compositions, as described herein, comprising an active compound and a pharmaceutically acceptable carrier or excipient of some sort, may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate-buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well-known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants, and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include but are not limited to, any non-toxic, inert solid, semisolid or liquid filler, diluent, encapsulating material, or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some aspects, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain aspects, the preservative is an anti-oxidant. In other aspects, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain aspects, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions, may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed, including synthetic mono- or diglycerides. In addition, fatty acids such as oleic acid are used in the preparation of injectables. In certain aspects, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter or by incorporating sterilizing agents in the form of sterile solid compositions, which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate-controlling membrane or by dispersing the particles in a polymer matrix or gel.

### Kits

Kits for practicing the methods described herein are further provided. By “kit” is intended any manufacture (e.g., a package or a container) comprising at least one reagent, e.g., any one of the compounds described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

Also disclosed are kits that comprise a composition comprising a compound disclosed herein in one or more containers. The disclosed kits can optionally include pharmaceutically acceptable carriers and/or diluents. In one aspect, a kit includes one or more other components, adjuncts, or adjuvants as described herein. In another aspect, a kit includes one or more anti-cancer agents, such as those agents described herein. In one aspect, a kit includes instructions or packaging materials that describe how to administer a compound or composition of the kit. Containers of the kit can be of any suitable material, e.g., glass, plastic, metal, etc., and of any suitable size, shape, or configuration. In one aspect, a compound and/or agent disclosed herein is provided in the kit as a solid, such as a tablet, pill, or powder form. In another aspect, a compound and/or agent disclosed herein is provided in the kit as a liquid or solution. In one aspect, the kit comprises an ampoule or syringe containing a compound and/or agent disclosed herein in liquid or solution form.

In view of the described compounds, compositions, and methods, hereinbelow are described certain more particular aspects of the disclosure. These particularly recited aspects should not, however, be interpreted to have any limiting effect on any different claims containing different or more general teachings described herein, or that the “particular” aspects are somehow limited in some way other than the inherent meanings of the language and formulae literally used therein.
Aspect 1. A method of treating a cancer in a subject in need thereof comprising administering to the subject a therapeutically effective amount of withaferin A and an immune checkpoint blocker.
Aspect 2. The method of aspect 1, wherein the cancer is resistant or has developed resistance to treatment with an immune checkpoint blocker alone or in combination with another therapeutic agent other than withaferin A, or wherein the cancer is susceptible to developing resistance to treatment with an immune checkpoint blocker alone or in combination with another therapeutic agent other than withaferin A.
Aspect 3. The method of aspect 1 or aspect 2, wherein the cancer is selected from basal cell carcinoma, bladder cancer, breast cancer, cervical cancer, cholangiocarcinoma, colorectal cancer, endometrial cancer, esophageal cancer, head and neck cancer, hepatocellular carcinoma, Hodgkin lymphoma, melanoma, mesothelioma, Merkel cell carcinoma, non-small cell lung cancer, renal cell carcinoma, small cell lung cancer, squamous cell carcinoma, stomach cancer, and urothelial carcinoma.
Aspect 4. The method of any one of aspects 1-3, wherein the immune checkpoint blocker comprises an inhibitor of CTLA-4.
Aspect 5. The method of aspect 4, wherein the immune checkpoint blocker comprises ipilimumab.
Aspect 6. The method of aspect 4, the immune checkpoint blocker comprises tremelimumab.
Aspect 7. The method of any one of aspects 1-3, the immune checkpoint blocker comprises an inhibitor of PD-1.
Aspect 8. The method of aspect 7, wherein the immune checkpoint blocker comprises pembrolizumab.
Aspect 9. The method of aspect 7, wherein the immune checkpoint blocker comprises nivolumab.
Aspect 10. The method of aspect 7, wherein the immune checkpoint blocker comprises cemiplimab.
Aspect 11. The method of aspect 7, wherein the immune checkpoint blocker comprises dostarlimab.
Aspect 12. The method of aspect 7, wherein the immune checkpoint blocker comprises retafanlimab.
Aspect 13. The method of aspect 7, wherein the immune checkpoint blocker comprises toripalimab.
Aspect 14. The method of aspect 7, wherein the immune checkpoint blocker comprises vopratelimab (JTX-4014).
Aspect 15. The method of aspect 7, wherein the immune checkpoint blocker comprises spartalizumab (PDR001).
Aspect 16. The method of aspect 7, wherein the immune checkpoint blocker comprises cambrelizumab (SHR1210).
Aspect 17. The method of aspect 7, wherein the immune checkpoint blocker comprises sintilimab (IBI308).
Aspect 18. The method of aspect 7, wherein the immune checkpoint blocker comprises tislelizumab (BGB-A317).
Aspect 19. The method of aspect 7, wherein the immune checkpoint blocker comprises INCMGA00012 (MGA012).
Aspect 20. The method of aspect 7, wherein the immune checkpoint blocker comprises AMP-224.
Aspect 21. The method of aspect 7, wherein the immune checkpoint blocker comprises AMP-514 (MEDI0680).
Aspect 22. The method of aspect 7, wherein the immune checkpoint blocker comprises acrixolimab (YBK-006).
Aspect 23. The method of any one of aspects 1-3, wherein the immune checkpoint blocker comprises an inhibitor of PD-L1.
Aspect 24. The method of aspect 23, wherein the immune checkpoint blocker comprises atezolizumab.
Aspect 26. The method of aspect 23, wherein the immune checkpoint blocker comprises avelumab.
Aspect 27. The method of aspect 23, wherein the immune checkpoint blocker comprises durvalumab.
Aspect 28. The method of aspect 23, wherein the immune checkpoint blocker comprises KN035.
Aspect 29. The method of aspect 23, wherein the immune checkpoint blocker comprises cosibelimab (CK-301).
Aspect 30. The method of aspect 23, wherein the immune checkpoint blocker comprises AUNP12.
Aspect 31. The method of aspect 23, wherein the immune checkpoint blocker comprises CA-170.
Aspect 32. The method of aspect 23, wherein the immune checkpoint blocker comprises BMS-986189.
Aspect 33. The method of any one of aspects 1-32, further comprising administering one or more additional therapeutic agents.
Aspect 34. The method of any one of aspects 1-33, further comprising administering a therapeutically effective amount of ionizing radiation.
Aspect 35. The method of any one of aspects 1-34, wherein the subject is a human. Aspect 36. A method of treating non-small cell lung cancer in a subject in need thereof comprising administering to the subject a therapeutically effective amount of withaferin A and a PD-L1 inhibitor.
Aspect 37. The method of aspect 36, wherein the PD-L1 inhibitor comprises atezolizumab.
Aspect 38. The method of aspect 36, wherein the PD-L1 inhibitor comprises avelumab.
Aspect 39. The method of aspect 36, wherein the PD-L1 inhibitor comprises durvalumab.
Aspect 40. The method of aspect 36, wherein the PD-L1 inhibitor comprises KN035.
Aspect 41. The method of aspect 36, wherein the PD-L1 inhibitor comprises cosibelimab (CK-301).
Aspect 42. The method of aspect 36, wherein the PD-L1 inhibitor comprises AUNP12.
Aspect 43. The method of aspect 36, wherein the PD-L1 inhibitor comprises CA-170.
Aspect 44. The method of aspect 36, wherein the PD-L1 inhibitor comprises BMS-986189.
Aspect 45. The method of any one of aspects 36-44, wherein the non-small cell lung cancer is resistant or has developed resistance to treatment with the PD-L1 inhibitor alone.
Aspect 46. The method of any one of aspects 36-45, wherein the subject is a human.

A number of aspects of the disclosure have been described. Nevertheless, it will be understood that various modifications may be made without departing from the spirit and scope of the invention. Accordingly, other aspects are within the scope of the following claims.

By way of non-limiting illustration, examples of certain aspects of the present disclosure are given below.

### Examples

The following examples are set forth below to illustrate the compounds, compositions, and methods disclosed and claimed herein, along with associated methods and results according to the disclosed subject matter. These examples are not intended to be inclusive of all aspects of the subject matter disclosed herein but rather to illustrate representative methods and results. These examples are not intended to exclude equivalents and variations of the present disclosure, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions, that can be used to optimize the product purity and yield obtained from the described process. Only reasonable and routine experimentation will be required to optimize such process conditions.

### Example 1. Withaferin A Increases the Effectiveness of Immune Checkpoint Blocker for the Treatment of Non-Small Cell Lung Cancer

Lung cancer is the leading cause of cancer-related deaths. Immunotherapy activates the patient's immune system to identify and kill cancer cells. Moreover, memory immune cells are formed that prevent the recurrence of cancer, leading to durable responses. However, only 20% of patients benefit from immunotherapy because the tumor-derived factors suppress the immune response. Herein, we tested if Withaferin A (an herbal compound) can make immunotherapy more effective in lung cancer patients. We found that Withaferin A induces the production of molecules from lung cancer cells that increase the infiltration of immune cells but are not able to kill cancer cells. Notably, in an immunocompetent mouse model of lung cancer, treatment with a combination of Withaferin A and an immunotherapy regimen showed more effectiveness than immunotherapy alone in activating immune cells and reducing tumor growth. This study presents a novel approach that can be tested clinically to improve lung cancer immunotherapy.

Treatment of late-stage lung cancers remains challenging with a five-year survival rate of 8%. Immune checkpoint blockers (ICBs) revolutionized the treatment of non-small cell lung cancer (NSCLC) by reactivating anti-tumor immunity. Despite achieving durable responses, ICBs are effective in only 20% of patients due to immune resistance. Therefore, synergistic combinatorial approaches that overcome immune resistance are currently under investigation. Herein, we studied the immunomodulatory role of Withaferin A (WFA)-a herbal compound-and its effectiveness in combination with an ICB for the treatment of NSCLC. Our in vitro results show that WFA induces immunogenic cell death (ICD) in NSCLC cell lines and increases expression of the programmed death ligand-1 (PD-L1). The administration of N-acetyl cysteine (NAC), a reactive oxygen species (ROS) scavenger, abrogated WFA-induced ICD and PD-L1 upregulation, suggesting the involvement of ROS in this process. Further, we found that a combination of WFA and α-PD-L1 significantly reduced tumor growth in an immunocompetent tumor model. Our results showed that WFA increases CD-8 T-cells and reduces immunosuppressive cells infiltrating the tumor microenvironment. Administration of NAC partially inhibited the anti-tumor response of the combination regimen. In conclusion, our results demonstrate that WFA sensitizes NSCLC to α-PD-L1 in part via activation of ROS.

Despite being well studied, the immunomodulatory properties of WFA and its role in modulating the effectiveness of ICB in NSCLC remain unexplored. We reasoned that WFA treatment can overcome tumor immune resistance and sensitize NSCLC to ICB therapy. To test this idea, we first investigated whether WFA could activate anti-tumor immunity by inducing ICD or/and altering IC molecule expression. We then tested the effectiveness of the combination treatment of WFA and α-PD-L1 as an ICB in an NSCLC immunocompetent mouse model. Our in vitro findings show that WFA-induced apoptotic ICD is associated with the DAMPs from the dying cells. Additionally, WFA altered the expression of IC molecules in NSCLC cell lines which sensitizes cancer cells to α-PD-L1 therapy. Importantly, we show for the first time that WFA sensitized an ICB-resistant tumor mouse model to α-PD-L1 therapy and restored anti-tumor immunity by altering tumor-immune infiltration and ROS levels in the TME in vivo.

### Materials and Methods

### Chemotherapeutic Drugs and Antibodies

WFA powder (5119-48-2) was purchased from ChromaDex Standards (Los Angeles, CA, USA) and reconstituted using Dimethyl Sulfoxide (DMSO) (Sigma-Aldrich, Burlington, MA, USA) to form 100 and 10 mM stock solutions, and stored at −20° C. in a freezer before use. For in vitro cell treatments, WFA stock solutions were diluted using complete culture media at the desired concentration (ranging from 10 μM to 0.4 μM). For in vivo treatments, WFA was diluted using Glyceryl Trioctanoate ((Sigma-Aldrich, Burlington, MA, USA) at the desired dose (4 mg/kg). In vivo mouse α-PD-L1 antibody (clone 10F.9G2) and rat IgG2b isotype control (clone LTF-2) were purchased from BioXcell (Lebanon, NH, USA). N-acetyl cysteine (NAC) was purchased from Sigma-Aldrich (Burlington, MA, USA) (A9165-25G) and L-Glutathione was purchased from Spectrum mfg. Corp (Gardena, CA, USA) (39G505). NAC or Glutathione were solubilized in PBS to make a 0.5 M solution and freshly prepared before each experiment. STATTIC (STAT3 inhibitor), PX-478 (HIFla inhibitor), and Brusatol (NRF-2 inhibitor) were all purchased from Selleck Chemicals (Houston, TX, USA) and solubilized in DMSO to create 10 mM stocks.

### Cell Lines

Human cell lines H1650, A549, HCT-116, MDA-MB-231 and mouse cell lines Lewis Lung Carcinoma (LLC) and 4T1 were purchased from the American Type Culture Collection (ATCC). The murine cell line MC-38 was provided by Dr. Shari Pilon-Thomas (Moffitt Cancer Center). H1650, A549, 4T1, and MDA-MB-231 cells were cultured in Roswell Park Memorial Institute (RPMI) media (Cytiva HyClone, Logan, UT, USA) supplemented with 10% heat inactivated fetal bovine serum (FBS) (Thermo Fisher Scientific, Waltham, MA, USA, 900-108) and 1% anti-bacterial/anti-mycotic solution (Cytiva HyClone, Logan, UT, USA, SV3007901). LLC cells were cultured in Dulbecco's Modified Eagle Medium media (Gibco, Grand Island, NY, USA) supplemented with 10% heat inactivated FBS and 1% antibacterial/anti-mycotic solution. The colorectal cancer cell line MC-38 was cultured in RPMI media supplemented with 2 mM L-glutamine (Gibco, Grand Island, NY, USA), 0.1 mM nonessential amino acids (Gibco, Grand Island, NY, USA), 1 mM sodium pyruvate (Gibco, Grand Island, NY, USA), 100 U/mL penicillin, 100 mg/mL streptomycin, and 10% heat inactivated FBS. The human colorectal cancer cell line HCT-116 was cultured in McCoy's 5A medium (Gibco, Grand Island, NY, USA) supplemented with 10% heat inactivated FBS and 1% anti-bacterial/anti-mycotic solution. Bone marrow-derived dendritic cells were cultured in complete RPMI media supplemented with 2 mM Glutamax (Gibco, Grand Island, NY, USA)), 50 μM β-mercaptoethanol, (Fisher Scientific, Waltham, MA, USA), and 20 ng of granulocyte monocyte colony stimulating factor (GM-CSF) (Pepro Tech, Cranbury, NJ, USA, 315-03) per ml. All cell lines were incubated at 37° C. in a humidified 5% CO₂ incubator.

### Cell Titer-Glo Assay

LLC, H1650, or A549 was plated in 96-well plates and treated with WFA (serialdilutions 10-0.078 μM) in triplicates. DMSO control was used to ensure that the observed cell death is due to WFA treatment only. Forty-eight hours after treatment, cell viability was determined using Cell Titer-Glo assay (Promega, Madison, WI, USA) according to the manufacturer's instructions. Luminescence was measured using a white well plate in a Bio-Tek Synergy H4 plate reader (Bio Tek, Winooski, VT, USA). Average luminescence normalized to untreated control cells was graphed against log WFA concentration and non-linear regression was performed to determine the inhibitory concentration 50 (IC50) value of WFA in each cell line.

### Annexin V Assay

NSCLC cell lines were treated with different concentrations of WFA for forty-eight hours then collected using Accutase cell detachment solution (Innovative Cell Technologies, San Diego, CA, USA). Apoptotic cell death was measured by flow cytometry using Propidium iodide or 40,6-diamidino-2-phenylindole (DAPI) viability dye and eBioscience Annexin V Apoptosis Detection Kit Allophycocyanin (APC) (Thermo Fisher Scientific, Waltham, MA, USA) according to the manufacturer's protocol.

### Flow Cytometry

In vitro WFA-treated or control cells were collected using Accutase cell detachment solution and stained with FACS-diluted (PBS, 10% FBS, 2 mMEDTA) pre-titrated fluorophore conjugated or primary unconjugated antibody solutions (Table 1) for 30 min on ice in the dark. In case of fluorophore-conjugated antibodies, samples are washed and immediately acquired after adding DAPI viability dye, while samples stained with unconjugated antibodies are incubated with the proper secondary fluorophore-conjugated antibody for 30 min before washing and acquisition. For in vivo experiments, the processed tumor or spleen single cell suspensions were used for surface staining with the pre-titrated antibodies (Table 1) for 30 min on ice in the dark. To exclude the dead cells from the analysis, a fixable live/dead Zombie Aqua viability kit (Biolegend, San Diego, CA, USA) was used according to the manufacturer's protocol. Samples were then fixed using 2% PFA for 15 min then washed and stored at 4° C. before acquisition. Samples were acquired using a Becton Dickenson Biosciences (BD, Franklin Lakes, NJ, USA) FACS Canto II or LSR II system at the University of South Florida COM Fred Wright Jr Flow Cytometry Core. Analysis was performed using FlowJo 8.7 software (BD, Franklin Lakes, NJ, USA).

TABLE 1
List of antibodies and reagents used in flow cytometry experiments.

| Antibody | Catalog # | Clone |
|---|---|---|
| Alexa Fluor 488 anti-mouse CD45 | 103121 | 30-F11 |
| Alexa Fluor 647 antimouse F4/80 | 123121 | BM8 |
| APC/Fire 750 antimouse/human CD11b | 101261 | M1/70 |
| BV421 cd80 | 104725 | 16-10A1 |
| Brilliant Violet 570 anti-mouse CD11c | 117331 | N418 |
| Brilliant Violet 650 anti-mouse CD206 (MMR) | 141723 | C068C2 |
| PE/Cy7 anti-mouse CD103 | 121425 | 2E7 |
| Brilliant Violet 605 anti-mouse CD8a | 100743 | 53-6.7 |
| Brilliant Violet 650 anti-mouse CD69 | 104541 | H1.2F3 |
| PE anti-mouse CD137 (4-1BB) | 106105 | |
| IgG (H + L) Cross-Adsorbed Secondary Antibody, | A11008 | Polyclonal |
| Alexa Fluor 488 | | |
| PE Rat UgG2b, κ Isotype Ctrl Antibody | 400607 | RTK4530 |
| PE Mouse IgG2b, κ Isotype Ctrl Antibody | 400311 | MPC-11 |
| OneComp eBeads Compensation Beads | 01-1111-41 | |
| PE/Dazzle 594 anti-mouse Ly-6G | 127647 | 1A8 |
| PE/Cy5 anti-mouse A/I-E | 107611 | M5/114.15.2 |
| PE anti-mouse CD86 | 159203 | A17199A |
| Alexa Fluor 488 anti-mouse NK-1.1 | 108717 | PK136 |
| Alexa Fluor 700 anti-mouse CD3 | 100215 | 17A2 |
| APC anti-mouse CD279 (PD-1) | 135209 | 29F.1A12 |
| Brilliant Violet 570 anti-mouse CD4 | 100541 | RM4-5 |
| Anti-Calreticulin antibody | Ab2907 | Polyclonal |
| PE/Cy5 anti-mouse CD25 | 102010 | PC61 |
| Alexa Fluor 700 anti-mouse Ly-6C | 128023 | HK1.4 |
| PE anti-mouse CD274 (B7-H1, PD-L1) Antibody | 124307 | 10F.9G2 |
| PE anti-human CD274 (B7-H1, PD-L1) Antibody | 329705 | 29E.2A3 |
| Zombie Aqua Fixable Viability Kit | 423101 | |

### HMGB-1 Assay

LLC cells were plated and treated with WFA or doxorubicin (ICD inducer) for 48 h. Supernatants were collected and centrifuged at 1000 RPM for 10 min. The levels of secreted HMGB-1 were measured using the HMGB-1 detection ELISA kit (Chondrex, 6010, Woodinville, WA, USA) according to the manufacturer's protocol.

### Ex Vivo Dendritic Cell Activation Assay

Myeloid progenitor cells were isolated from the femurs of C57BL/6 mice as described previously [37]. The isolated cells were cultured in RMPI media supplemented with GMCSF and refed as necessary. On day 8, the differentiated DCs (loosely attached) were collected, counted, and added to WFA-pretreated LLCs or untreated control LLCs at a ratio of 5:1 of DC: LLC cells. Twenty-four hours post co-culture, the DC were collected and stained with anti-CD11c, anti-CD80, anti-CD86, and anti-MHCII antibodies (BioLegend, San Diego, CA, USA). Then the samples were acquired using a BD LSR II flow cytometer and the data were analyzed using FlowJo 8.7 software (BD, Franklin Lakes, NJ, USA).

### Reverse Transcription and Quantitative PCR

Total cellular RNA was isolated from collected cell pellets using TRIzol reagent (Invitrogen, Waltham, MA, USA) and then quantified using the 2000 Nanodrop spectrophotometer (Thermo Fisher Scientific, Waltham, MA, USA). cDNA synthesis was performed using the Verso cDNA synthesis kit (Thermo Fisher Scientific, Waltham, MA, USA). qPCR was then performed using Forget-Me-Not EvaGreen qPCR Master Mix (Biotium, Fremont, CA, USA) in a Bio-Rad CFX384 thermocycler according to the manufacturer's instructions. The CFX Maestro software version 2.3 (Bio-Rad, Hercules, CA, USA) was used to calculate the ΔCt and ΔΔCt values and gene expression was normalized to housekeeping genes glyceraldehyde 3-phosphate dehydrogenase (GAPDH) or β-actin. The sequences of PCR primers used are listed in Table 2 (Integrated DNA Technologies, Inc, Coralville, IA, USA).

TABLE 2
List of primer sequences used in qRT-PCR.

| Gene | | |
|---|---|---|
| name | Species | Sequence |
| Pd-11 | Mouse | 5′-GTGAAACCCTGAGTCTTATCC-3′ |
| | | (SEQ ID NO: 1) |
| | | 5′-GACCATTCTGAGACAATTCC-3′ |
| | | (SEQ ID NO: 2) |
| PD-L1 | Human | 5′-TGCCGACTACAAGCGAATTACTG-3′ |
| | | (SEQ ID NO: 3) |
| | | 5′-CTGCTTGTCCAGATGACTTCGG-3′ |
| | | (SEQ ID NO: 4) |
| Bax | Mouse | 5′-GCGTGGTTGCCCTCTTCTACTTTG-3′ |
| | | (SEQ ID NO: 5) |
| | | 5′-AGTCCAGTGTCCAGCCCATGATG-3′ |
| | | (SEQ ID NO: 6) |
| BAX | Human | 5′-TCCAGGATCGACCAGGGCGA-3′ |
| | | (SEQ ID NO: 7) |
| | | 5′-AAAAGGGCGACAACCCGGCC-3′ |
| | | (SEQ ID NO: 8) |
| Bcl-2 | Mouse | 5′-GTCCCGCCTCTTCACTCAG-3′ |
| | | (SEQ ID NO: 9) |
| | | 5′-GATTCTGGTGTTTCCCCGTTGG-3′ |
| | | (SEQ ID NO: 10) |
| BCL-2 | Human | 5′-CTCCCTCTCCCCGCCACTCC-3′ |
| | | (SEQ ID NO: 11) |
| | | 5′-GGGGGTGTCTTCAATCACGCG-3′ |
| | | (SEQ ID NO: 12) |
| Bak-1 | Mouse | 5′-AAGGTGGGCTGCGATGAGTCC-3′ |
| | | (SEQ ID NO: 13) |
| | | 5′-GGGTCTCCTGCTGGTC-3′ |
| | | (SEQ ID NO: 14) |
| BAD | Human | 5′-GGGATGGGGGAGGAGCCCAG-3′ |
| | | (SEQ ID NO: 15) |
| | | 5′-AAGGTCACTGGGAGGGGGCG-3′ |
| | | (SEQ ID NO: 16) |
| IFNα | Mouse | 5′-GGACTTTGGATTCCCGCAGGAGAAG-3′ |
| | | (SEQ ID NO: 17) |
| | | 5′-GCTGCATCAGACAGCCTTGCAGGTC-3′ |
| | | (SEQ ID NO: 18) |
| IFNα | Human | 5′-GACTTCATCTTGGCTGTGA-3′ |
| | | (SEQ ID NO: 19) |
| | | 5′-TGATTTCTGCTCTGACAACCT-3′ |
| | | (SEQ ID NO: 20) |
| GAPDH | Mouse | 5′-CCTGGTATGACAATGAATACGGC-3′ |
| | | (SEQ ID NO: 21) |
| | | 5′-CTCCTTGGAGGCCATGTAGG-3′ |
| | | (SEQ ID NO: 22) |
| GAPDH | Human | 5′-CGACAGTCAGCCGCATCTT-3′ |
| | | (SEQ ID NO: 23) |
| | | 5′-CCCCATGGTGTCTTCAATCACGCG-3′ |
| | | (SEQ ID NO: 24) |
| B-actin | Mouse | 5′-AAATCTGGCACCACACCTTC-3′ |
| | | (SEQ ID NO: 25) |
| | | 5′-GGGGTGTTGAAGGTCTCAAA-3′ |
| | | (SEQ ID NO: 26) |
| B-ACTIN | Human | 5′-TTCTACAATGACCTGGGTGTG-3′ |
| | | (SEQ ID NO: 27) |
| | | 5′-GGGGTGTTGAAGGTCTCAAA-3′ |
| | | (SEQ ID NO: 28) |

### Protein Extraction and Western Blot

Total protein was isolated using Radioimmunoprecipitation Assay (RIPA) buffer (Thermo Fisher Scientific, Waltham, MA, USA) and sonication of cell pellets using Branson digital sonifier 450. The lysates were centrifuged at 16,000 rpm for 20 min and the supernatants containing total cellular proteins were collected. Then, protein levels were quantified using a Pierce Coomassie Protein assay kit (Thermo Fisher Scientific, Waltham, MA, USA) according to the manufacturer's instruction and the absorbance values at 595 nm were measured using a plate reader (Bio Tek, Winooski, VT, USA) and used to calculate protein concentrations. Fifty micrograms of protein were resolved by SDS-polyacrylamide gel electrophoresis (Bio-Rad, Hercules, CA, USA) and transferred to a 0.2 μm nitrocellulose membrane (Bio-Rad, Hercules, CA, USA) at 80 V for 2 h. Blots were blocked using 5% milk in TBS-T or 5% bovine albumin serum in the case of phosphoproteins for 1 h at room temperature. Blots were then incubated with the primary antibody (concentration 1:1000) in the blocking solution at 4° C. overnight with gentle agitation. Blots were washed with TBS-T three times and incubated with the secondary HRP-conjugated antibodies for an hour at room temperature. Primary and secondary antibodies used for western blotting are listed in Table 3. Protein bands were detected using SuperSignal West Pico PLUS Chemiluminescent Substrate or West Femto Maximum Sensitivity Substrate (Thermo Fisher Scientific, Waltham, MA, USA) and imaged using the ChemiDoc XRS imaging system (Bio-Rad, Hercules, CA, USA) according to the manufacturer's instructions. Band density was measured using Image J software version 1.54d and was normalized to housekeeping proteins (0-actin or Vinculin). Protein levels in the treatment groups were normalized to the untreated control and an average of 2 independent experiments ±SEM are shown in the bar graphs.

TABLE 3
List of antibodies used in western blotting.

| Antibody | Catalog number |
|---|---|
| Beta Actin antibody | SC-47778 |
| Vinculin antibody | SC-25336 |
| Mouse anti-rabbit IgG-HRP | SC-516102 |
| m-IgGκ BP-HRP | SC-516102 |
| Phospho-eIF2α (Ser51) D9G8) XP Rabbit | 3398 |
| mAb | |
| PD-L1/CD274 Rabbit pAb | A1645 |
| PD-L1 (E1L3N) XP Rabbit mAb | 13684 |
| CHOP (L63F7) Mouse mAb | 2895 |
| eIF2α (D7D3) CP Rabbit mAb | 5324 |
| Nrf2 Polyclonal Antibody | PA5-88084 |

### Ingenuity Pathway Analysis (IPA)

To identify the downstream regulators that contribute to WFA-induced PD-L1 upregulation, we constructed a gene interaction network between the PD-L1 signaling pathway and ROS (nitric oxide, oxygen radical, hydrogen peroxide, and lipoxygenase) signaling using the known molecular connections obtained from the IPA database (Qiagen, Redwood City, CA, USA). Fold change data from MCF-7 breast cancer cells treated with WFA (700 nM for 72 h) were obtained from the NCBI Gene Omnibus Expression (GEO) database (Series GSE53049) and overlaid onto the network to yield predictions of activation/inhibition in the IPA software version 90348151 (Qiagen, Redwood City, CA, USA).

### SiRNA Transfection

Cells were transfected using the TransIT-TKO transfection kit (Mirus Bio, Madison, WI, USA) according to the manufacturer's instructions. Briefly, LLC or H1650 cells were seeded in 24-well plates and incubated for 24 h. The transfection reagent was prepared by mixing NRF-2 or non-targeting siRNA with TransIT-X2 reagent in antibiotic-free, serum free Opti-MEM (Gibco, Grand Island, NY, USA) medium to reach a final concentration of 25 nM. The mixture was added to the cells for twenty-four hours to allow transfection. Subsequently, the cells were treated with WFA and incubated for twenty-four hours before collection. Non-targeting siRNA, mouse and human NRF-2 siRNA SMART pools were purchased from Dharmacon Reagents (Lafayette, CO, USA, D-001810-01-20, L-003755-00-0010 and L-040766-00-0010).

### Animal Studies

C57BL/6 mice were purchased from the Jackson Laboratory (Bar Harbor, ME, USA) and acclimated for a week. To establish the LLC syngeneic tumors, C57BL/6 mice were injected with a half million cells on the right flank and monitored until tumors were first palpable (2-3 mm diameter). Mice were then randomized into treatment groups (n=5 per group), to receive either vehicle (10% DMSO, 90% Glyceryl Trioctanoate), WFA (4 mg/kg), α-PD-L1(200 μg/mouse), isotype antibody (200 μg/mouse), NAC (100 mg/kg), a combination of α-PD-L1 and WFA, or a combination of WFA+α-PD-L1 and NAC intraperitonially. Treatments were administered every other day with a total of five treatments and mice body weights and tumor volumes were monitored using a caliper (Fisher Scientific, Waltham, MA, USA, 15-077-957). Tumor volumes (mm³) were calculated using the equation (length² x width/2), where the length is defined as the bigger tumor dimension. When the control tumors reached 10 mm×10 mm, mice were euthanized using CO₂ euthanasia; then, tumors and spleen were collected for downstream analyses. Briefly, spleens and tumors were collected and dissociated mechanically or using the Miltenyi Biotec mouse tumor dissociation kit (130-096-730) and filtered through 70 μm strains (Thermo Fisher Scientific, Waltham, MA, USA). Red blood cells were lysed using the ACK lysing buffer (Thermo Fisher Scientific, Waltham, MA, USA). Single cell suspensions were then used for flow cytometry.

### Statistical Analysis

Each experiment was repeated at least twice, and the results are represented as mean ±the standard error of the mean, SEM. To calculate statistical significance, an unpaired Student's t-test was used when comparing two groups, while Analysis of Variance (ANOVA) was used when comparing multiple groups. The Fisher LSD post hoc test was performed to compare the means of the treatment groups to that of the control group. Analyses were carried out and graphs were plotted using GraphPad Prism 8.0.1. * or #p<0.05, ** or ##p<0.01, *** or ###p<0.001, * * ** p<0.0001.

### Results

### WFA Induces ER-Stress Mediated Apoptosis in NSCLC

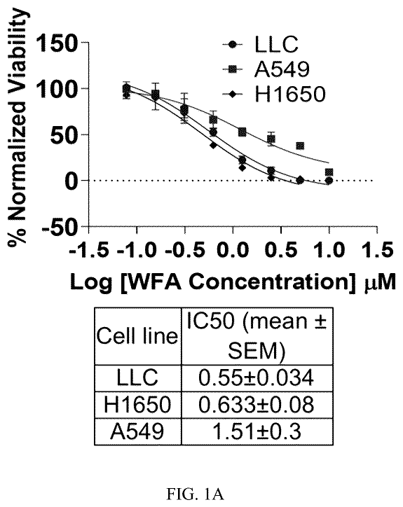

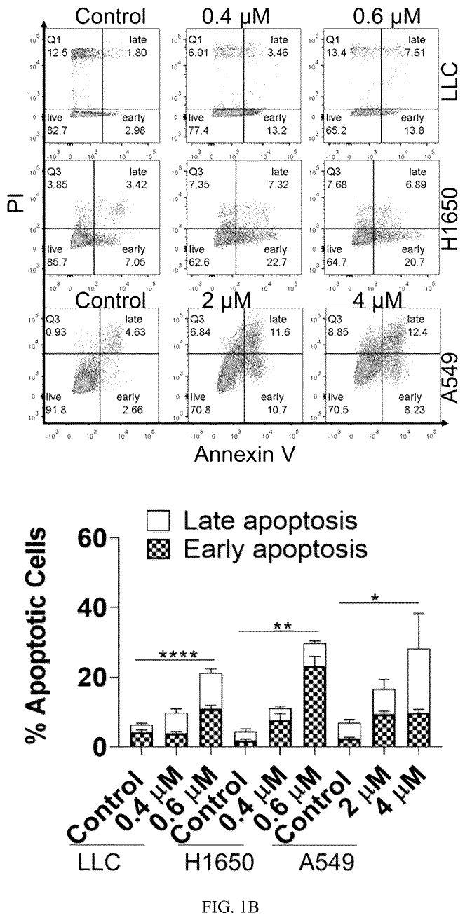

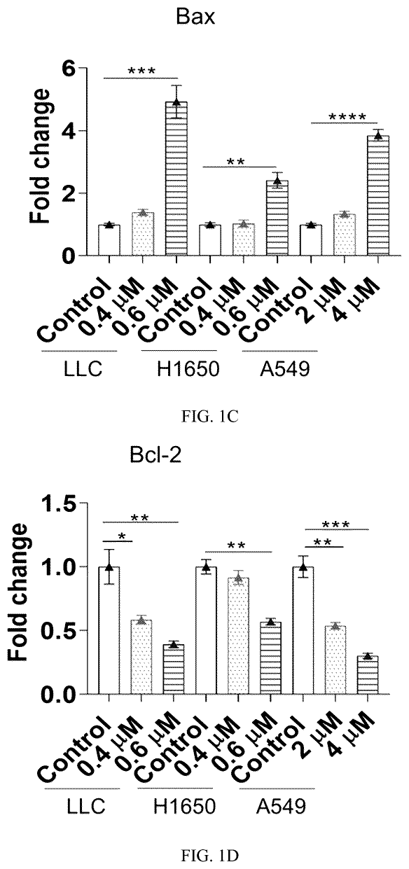

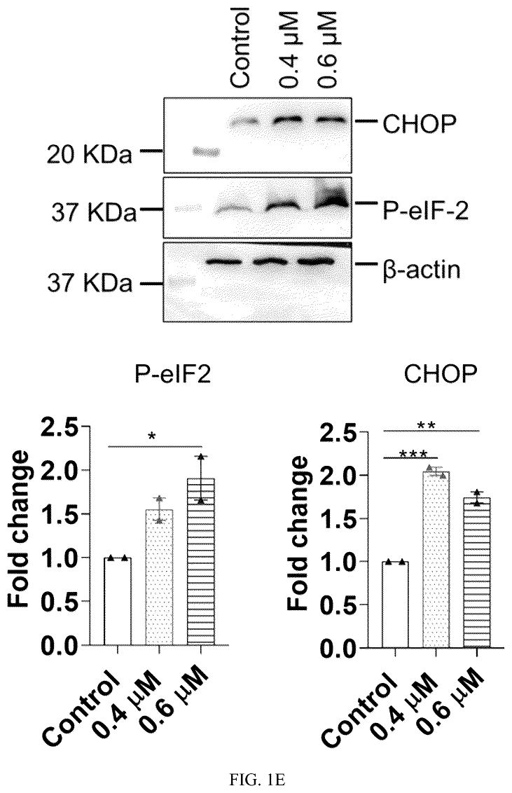

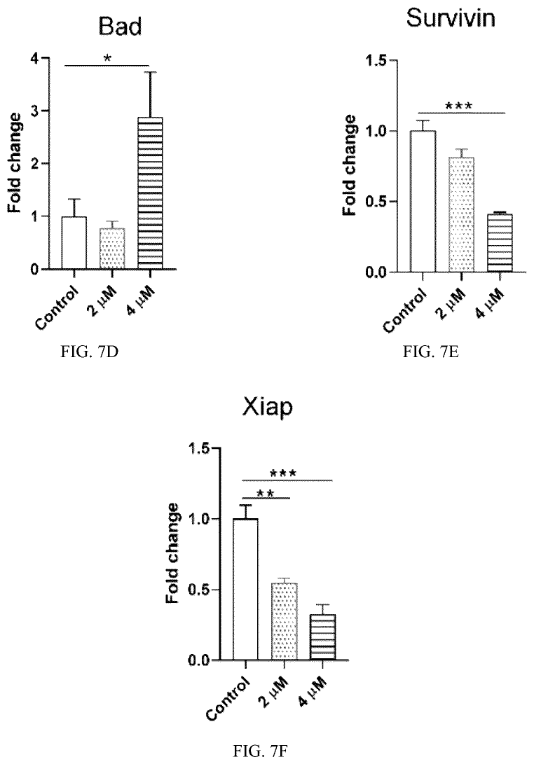

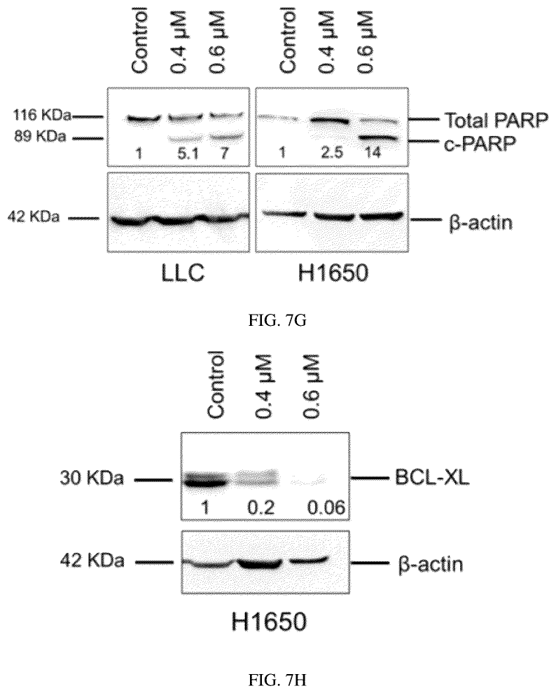

Despite being originally considered non-immunogenic, recent findings show that certain apoptotic triggers can induce ICD. For example, stressors that induce prolonged ER stress and unfolded protein response can lead to immunogenic apoptotic cell death [38]. Previously, WFA was shown to induce apoptotic cell death in NSCLC cell lines [33]. However, it is unclear whether it can activate the ER stress pathway and ICD. To confirm the mechanism by which WFA targets NSCLC, we treated the cell lines (LLC, H1650 and A549) with different concentrations of WFA for forty-eight hours. Cell Titer-Glo assay showed that WFA was toxic in these cell lines with IC50 values ranging from 0.5 to 1.5 μM (FIG. 1A). Consequently, we performed an annexin V assay that showed a significant increase in early and late apoptotic cell populations in WFA-treated cells compared to their untreated counterparts (FIG. 1). To investigate the molecular mechanisms of WFA-induced apoptosis, we measured the changes in both pro and anti-apoptotic marker transcripts using qRT-PCR and Western blotting. Our PCR studies revealed that WFA increased the transcription of the pro-apoptotic Bax protein transcripts (FIG. 1C), Bak-1 (FIG. 7A), and BAD (FIG. 7B). Moreover, WFA treatment reduced the transcripts of the anti-apoptotic proteins Bcl-2 (FIG. 1D), Xiap, and Survivin (FIGS. 7B, 7C, 7E, 7F). Moreover, western blotting showed that WFA increased PARP cleavage in LLC and H1650 cells and reduced the levels of BCL-XL in H1650 cells (FIGS. 7G, 7H). We then investigated whether WFA induces ER stress in NSCLC cell lines by quantifying the levels of ER stress markers using western blotting. We found that WFA increases the levels of C/EBP homologous protein (CHOP) and the phosphorylation of the eukaryotic initiation factor eIF-2 (FIGS. 1E-1F). In summary, we identified that WFA induces ER-stress mediated apoptosis in NSCLC cell lines.

### WFA Induces ICF in NSCLC Cell Lines

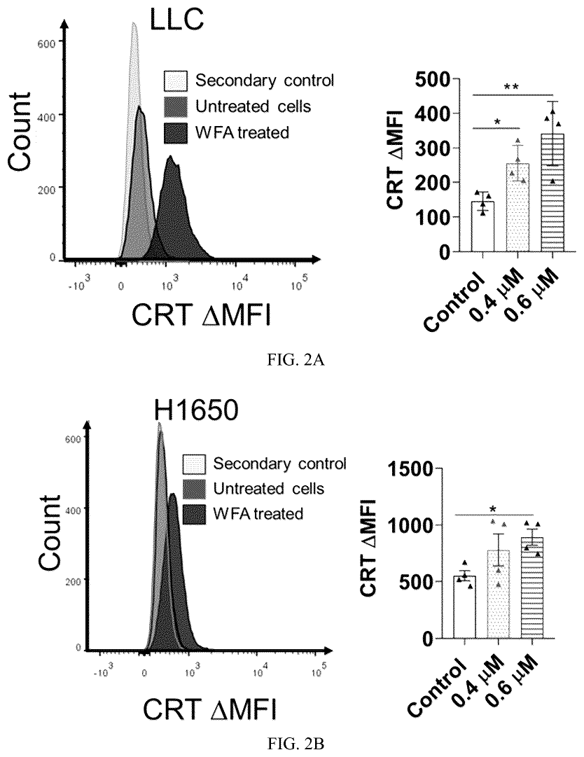

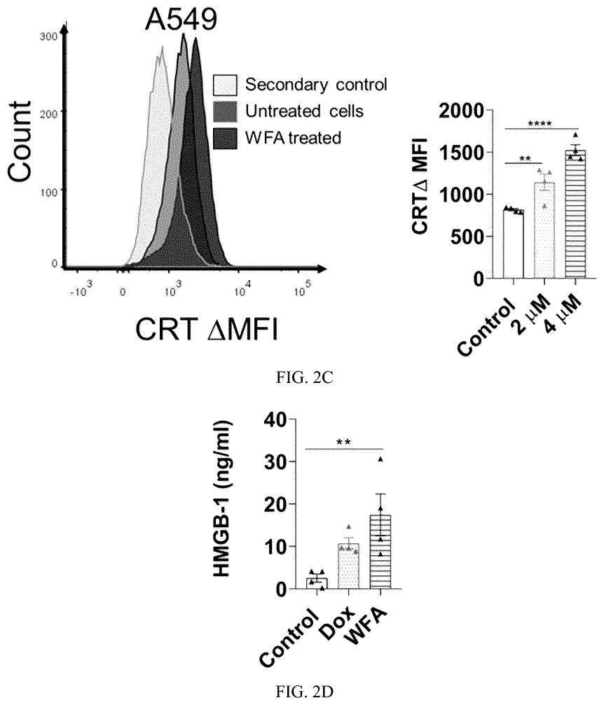

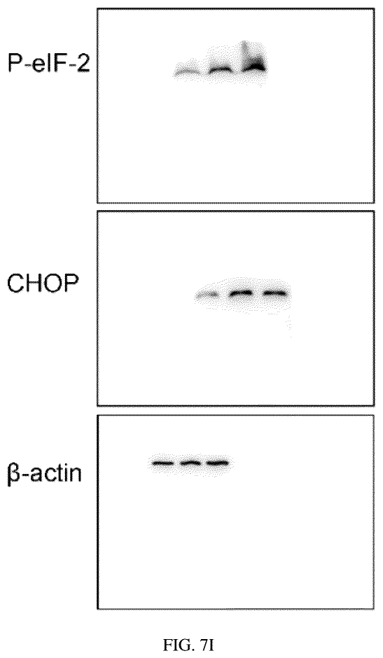

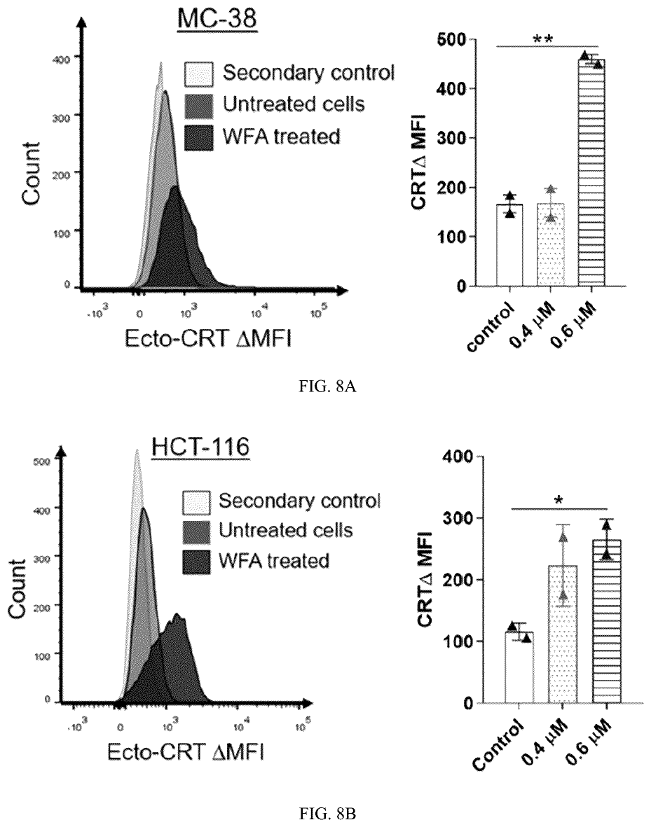

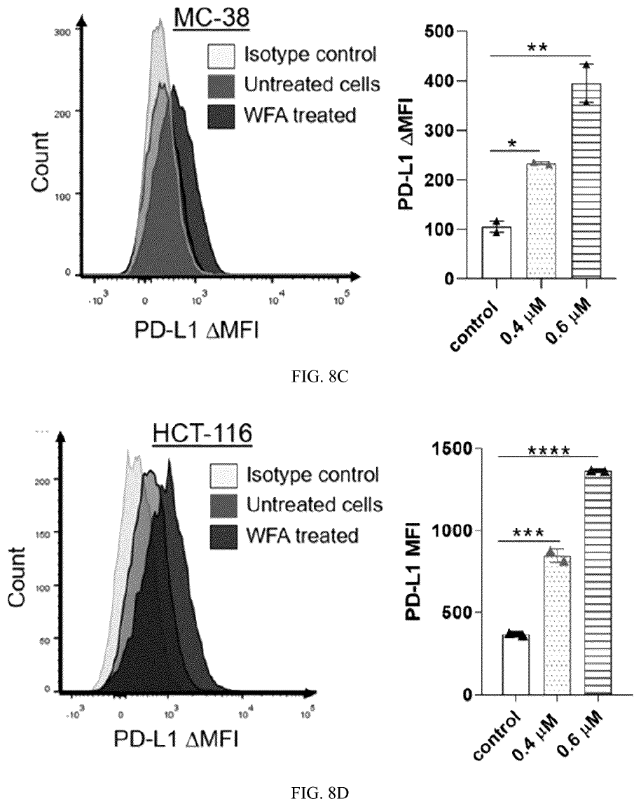

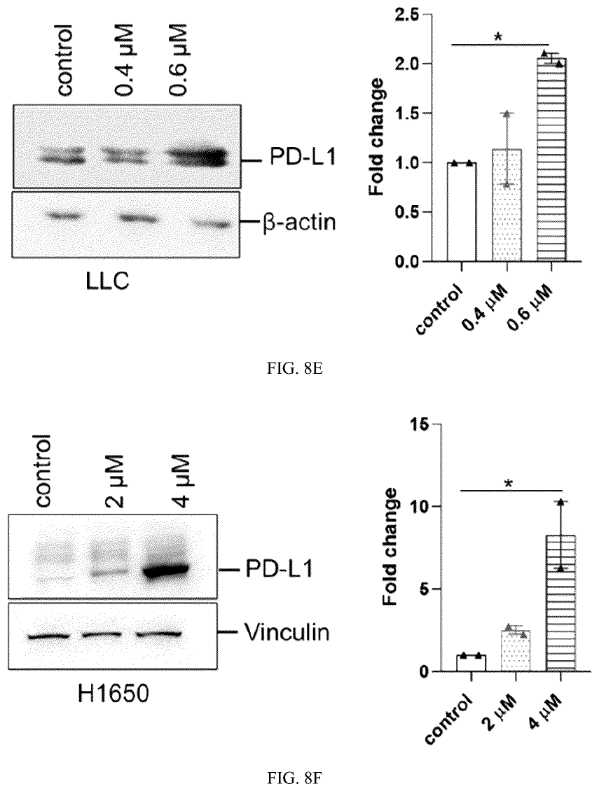

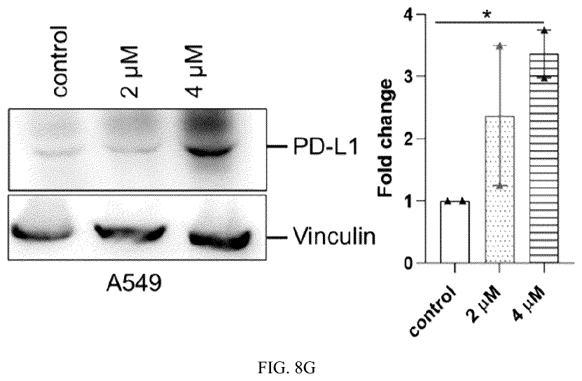

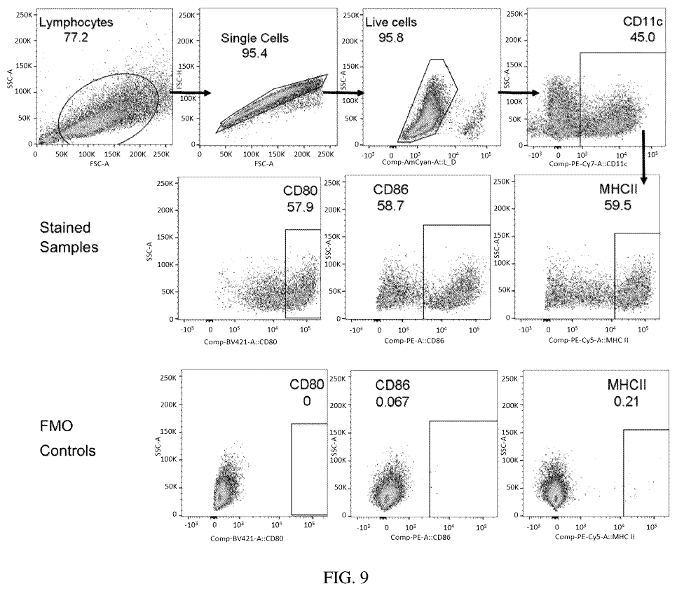

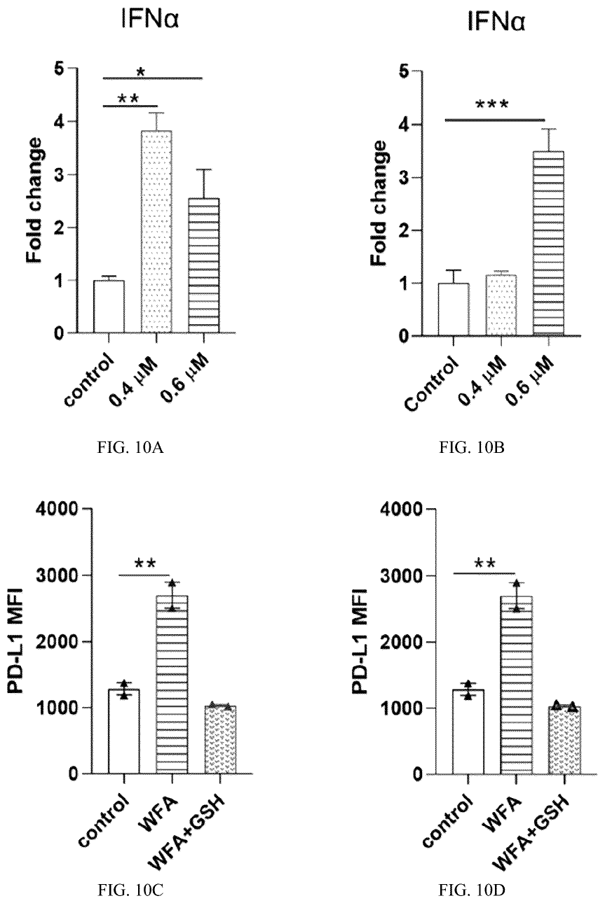

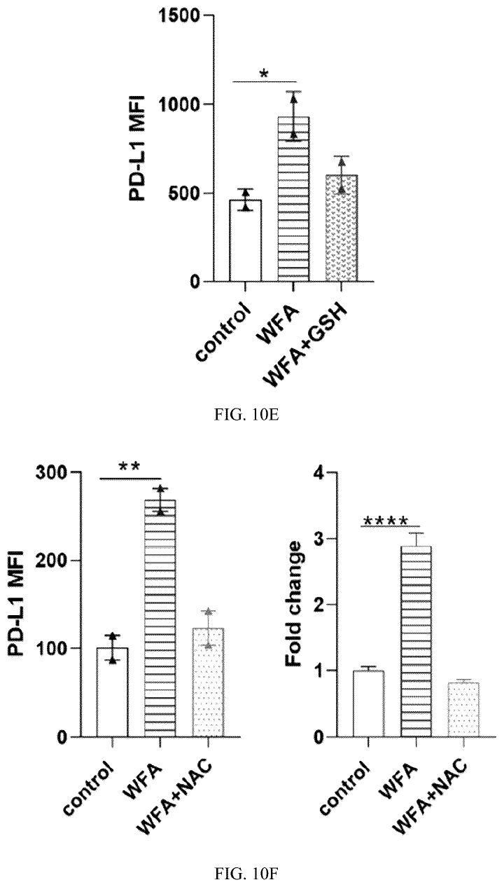

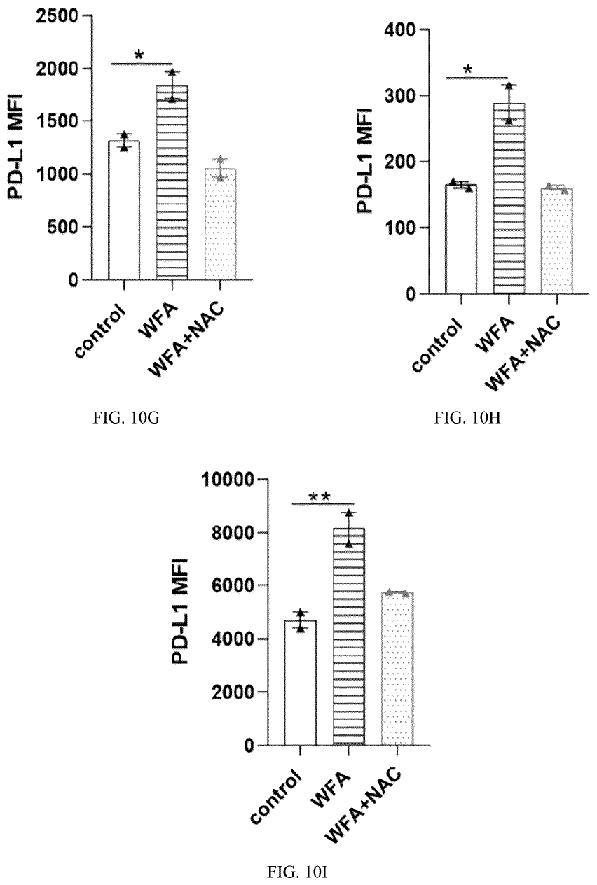

ICD is characterized by the expression or release of DAMPs such as CRT and HMGB-1 that act as a (eat me) signal to activate APCs [39]. Consequently, active APC can present tumor antigens along with the stimulatory signals essential to produce an anti-tumor T-cell response [40]. To determine if WFA can induce ICD in lung cancer, we treated LLC, H1650 and A549 cells with WFA and then collected the cells or supernatants to measure the levels of cell surface expressed CRT (ecto-CRT) or secreted HMGB-i, respectively. We found that WFA treatment increased ecto-CRT expression in LLC, H1650 cells and A549 cells (FIGS. 2A-2C) by flow cytometry analysis. These findings prompted us to test if WFA can also induce ICD in other types of cancer (e.g., colorectal cancer). To test this hypothesis, we treated the murine MC38 and human HCT-116 colorectal cancer cell lines with WFA and then measured the change in ecto-CRT levels using flow cytometry. Interestingly, WFA increased the levels of ecto-CRT in both MC38 and HCT-116 cell lines (FIGS. 8A-8B). To measure the levels of secreted HMGB-i, we collected the supernatants of WFA-treated LLC cells and centrifuged them to remove any dead cells or debris. Using the HMGB-1 ELISA kit, we found that WFA treatment increased the HMGB-1 levels secreted in the supernatants of WFA-treated LLC cells (FIG. 2D). To confirm that the release of DAMPs by WFA treated cells can activate APCs, we isolated bone marrow-derived myeloid progenitors from C57BL/6 mice and allowed their differentiation into DCs (day 8). We then collected the bone marrow-derived DCs and co-cultured them with WFA-pretreated LLC for twenty-four hours. Subsequently, DCs were collected and their expression of the activation markers CD80, CD86, and MHC-II was measured using flow cytometry (FIG. 9). Our results indicate that DCs co-cultured with WFA-treated cells expressed higher levels of the activation markers CD80, CD86, and NMC-II than those cultured with untreated controls (FIG. 2E). Moreover, qRT-PCR shows that WFA increased IFN-α, characteristic of ICD in LLC and H1650 cells [41] (FIGS. 10A-10B). Our findings confirm that WFA can induce ICD in NSCLC cells and colorectal cancer cells by promoting the release/expression of DAMPs which consequently activate APC.

### WFA Increases PD-L1 Expression in NSCLC Cell Lines

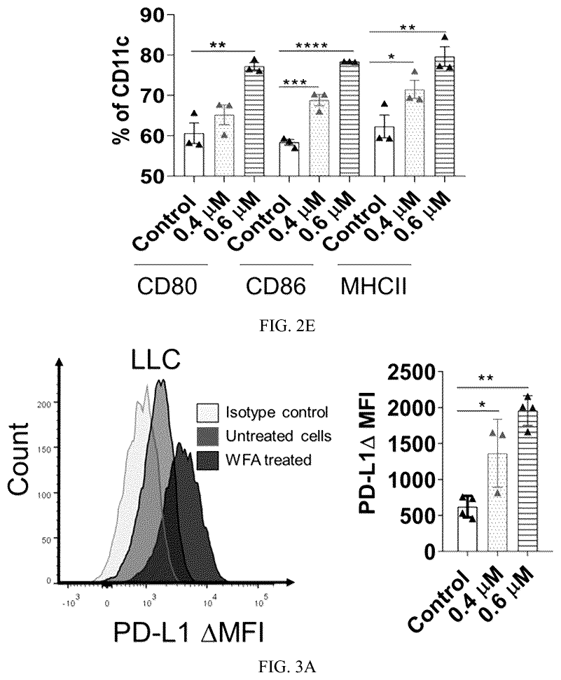

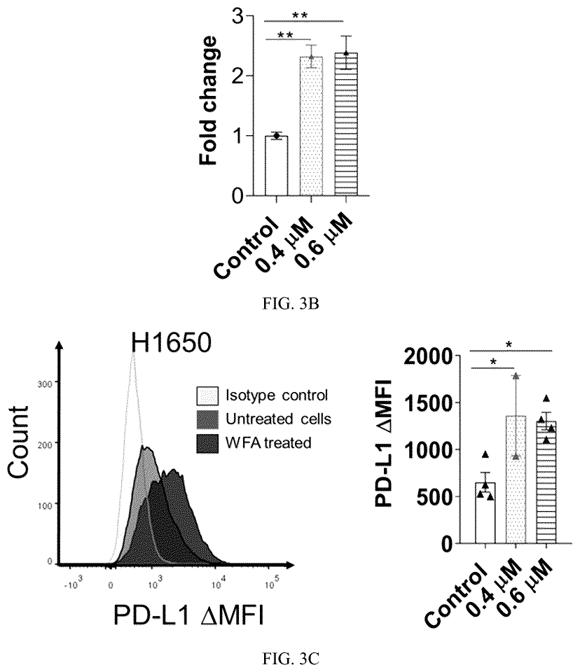

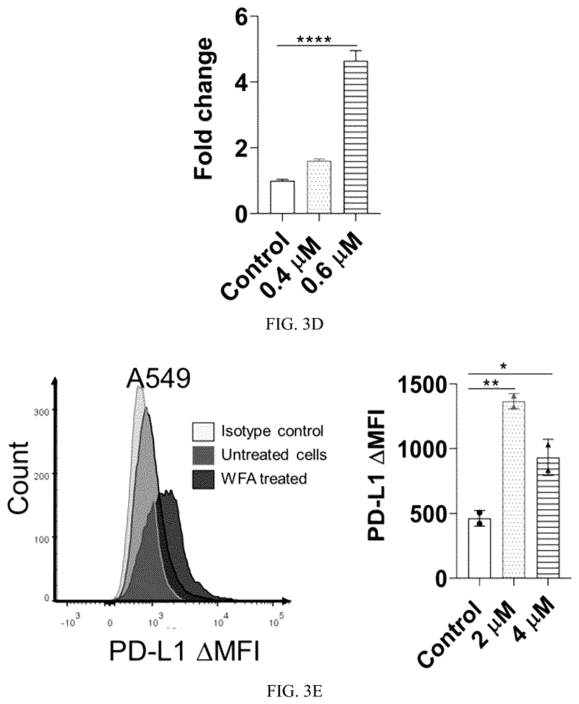

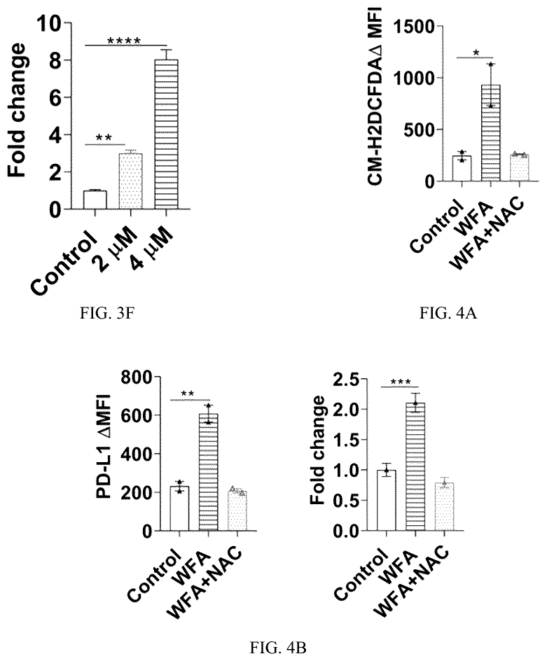

Due to the important role of PD-L1 as a biomarker of response to ICB therapy, we investigated the effect of WFA on PD-L1 expression in NSCLC cell lines. Towards this goal, we collected WFA-treated NSCLC cell lines to measure PD-L1 surface expression using flow cytometry. We found that WFA treatment consistently increased PD-L1 expression in LLC, H1650, and A549 cell lines (FIGS. 3A, 3C, 3E). Consistently, qRT-PCR (FIGS. 3B, 3D, 3F) and western blotting (FIGS. 8E-8G) confirmed that WFA induced PD-L1 expression in NSCLC cell lines. We then investigated whether WFA can also increase PD-L1 expression in other types of cancers. Similarly, WFA increased PD-L1 expression in MC38, HCT116 colon cancer cell lines (FIGS. 8C-8D). In conclusion, WFA upregulated PD-L1 expression in NSCLC and colorectal cancer cell lines which can sensitize these tumors to ICBs.

### WFA Mediates PD-L1 Upregulation and ICD by Increasing ROS Production

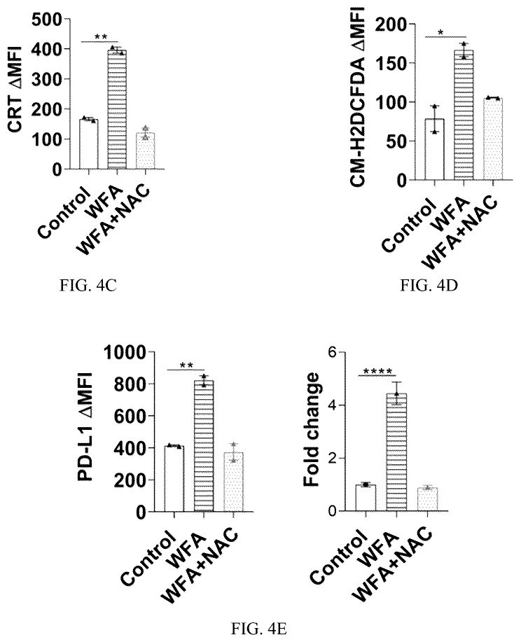

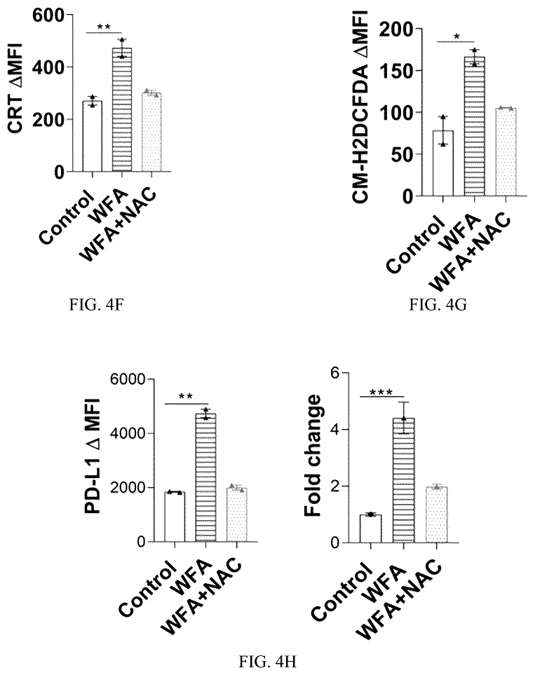

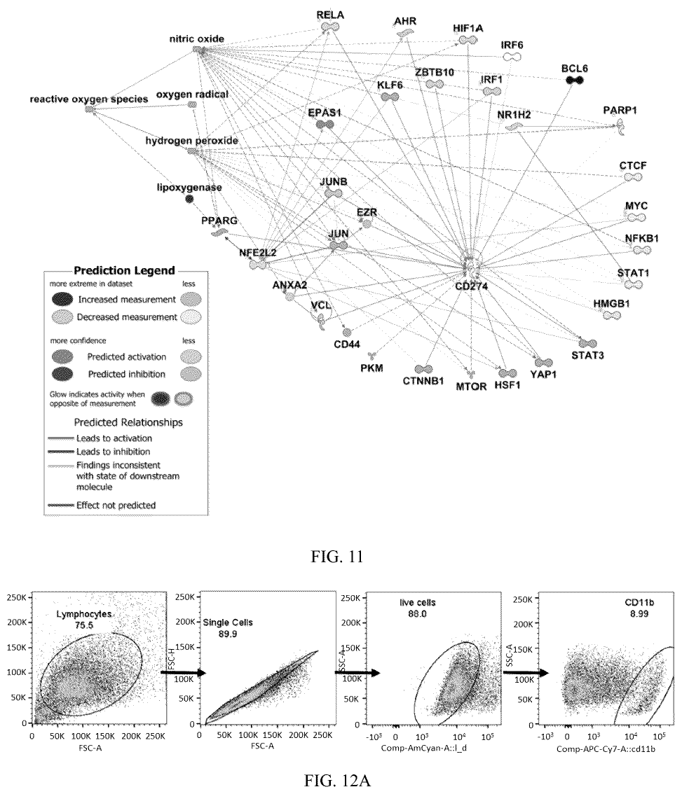

As our findings show that WFA induces PD-L1 upregulation and ICD in NSCLC, and colon cancer cell lines, we investigated the mechanism responsible for WFA-mediated PD-L1 expression changes. Others have shown that WFA induces cancer cell death mainly through increased ROS production which can be abrogated by ROS scavengers (e.g., NAC or Glutathione) [33,42]. Due to the central role of ROS in WFA-mediated cytotoxicity, we investigated if ROS production plays a role in PD-L1 upregulation as well. To test our hypothesis, we treated NSCLC cell lines with WFA, NAC, or a combination of both. To confirm the changes in ROS levels with each treatment, we stained these cells with ROS indicator CM-H2DCFDA and found that WFA increased ROS levels while NAC reduced them (FIGS. 4A, 4D, 4G). Interestingly, NAC treatment completely reversed the WFA-mediated PD-L1 increase as measured by both flow cytometry and qRT-PCR (FIGS. 4B, 4E, 4H). Moreover, NAC completely reversed ecto-CRT expression in NSCLC cell lines (FIGS. 4C, 4F, 4I). To confirm, we used Glutathione (GSH) as a ROS scavenger and found that it abrogated PD-L1 expression as well (FIGS. 10C-10E). In addition, we examined the effects of NAC in WFA treated colorectal and breast cancer cell lines. Similar to NSCLC cell lines, we found that NAC completely reversed PD-L1 upregulation in MC-38, HCT-116, 4T-1, and MDA-MB-231 cell lines (FIGS. 10F-10I, respectively). To investigate the downstream regulators that may be involved in ROS-mediated PD-L1 upregulation, we used IPA analysis of a publicly available dataset of WFA-treated MCF-7 breast cancer cells (Series GSE53049). First, we constructed a network showing the molecular connection between PD-L1 and ROS (nitric oxide, oxygen radical, hydrogen peroxide, and lipoxygenase). Subsequently, we overlaid the gene expression data obtained from the dataset onto the molecular network to predict the pathways involved in WFA-mediated PD-L1 upregulation (FIG. 11). IPA predictions show that a complex network of multiple downstream regulators may be involved in PD-L1 upregulation. To validate the IPA results, we treated NSCLC cells with WFA or a combination of WFA and STATTIC (STAT-3 inhibitor), PX-478 (HIF1α inhibitor), or brusatol (NRF-2 inhibitor). Flow cytometry showed that neither STATTIC nor PX-478 were able to reverse WFA-induced PD-L1 upregulation (FIGS. 13A-13C). However, adding brusatol-an NRF-2 inhibitor-to WFA treated cells completely abrogated the upregulation of PD-L1 in LLC, H1650, and A549 cells (FIGS. 13D-13F). To confirm the involvement of NRF-2, we transfected LLC or H1650 with siRNA for NRF-2 or scramble and then treated these cells with WFA. We used western blotting to confirm that NRF-2 siRNA indeed inhibits the activation of NRF-2 (FIG. 13I). Unlike our previous findings, we found that WFA still upregulated PD-L1 in NRF-2 knockdown cells similar to the scramble controls (FIG. 13G-13H). Due to the discrepancy in our findings between brusatol and NRF-2 KO and the lack of specificity of the pharmacologic inhibitors, we conclude that NRF-2 may not be essential in WFA-induced PD-L1 upregulation. Further studies are needed to elucidate the downstream regulators of WFA mediated PD-L1 upregulation. In summary, we conclude that WFA mediates PD-L1 and ecto-CRT expression mainly by inducing ROS production in vitro.

### WFA Sensitized LLC Syngeneic Mouse Tumors to α-PD-L1 In Vivo

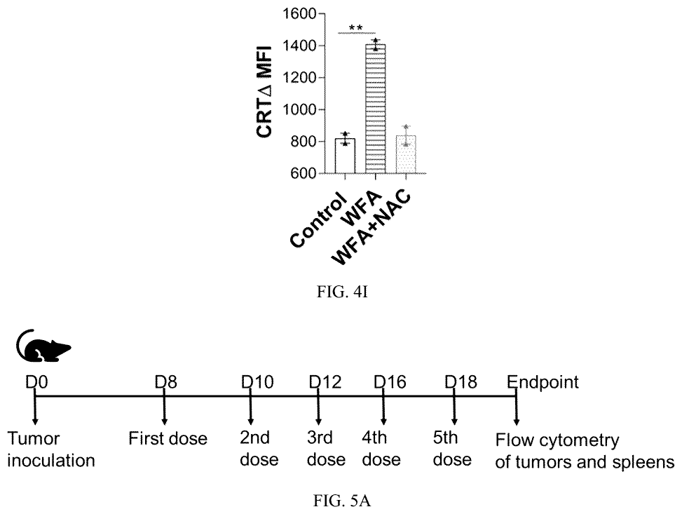

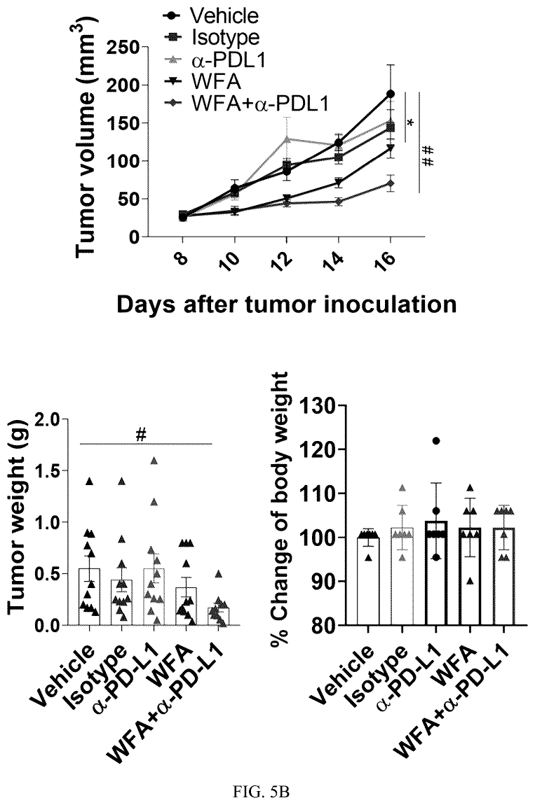

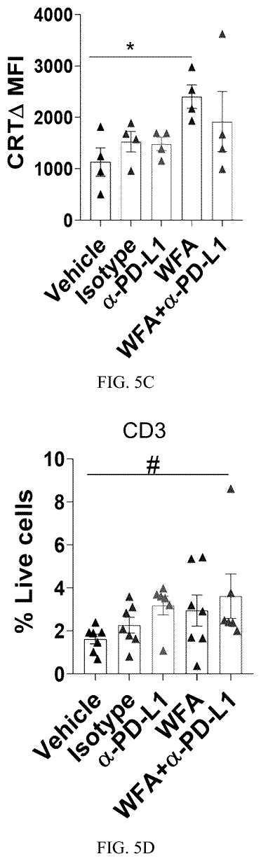

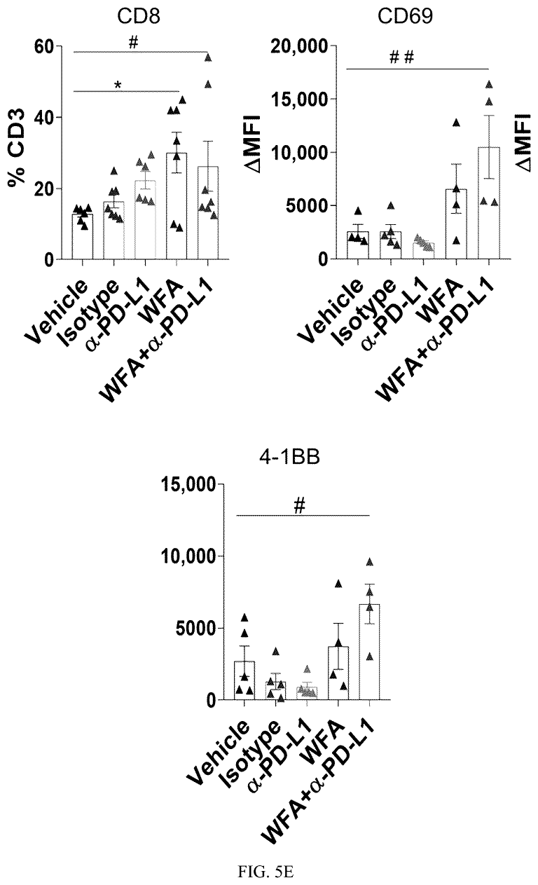

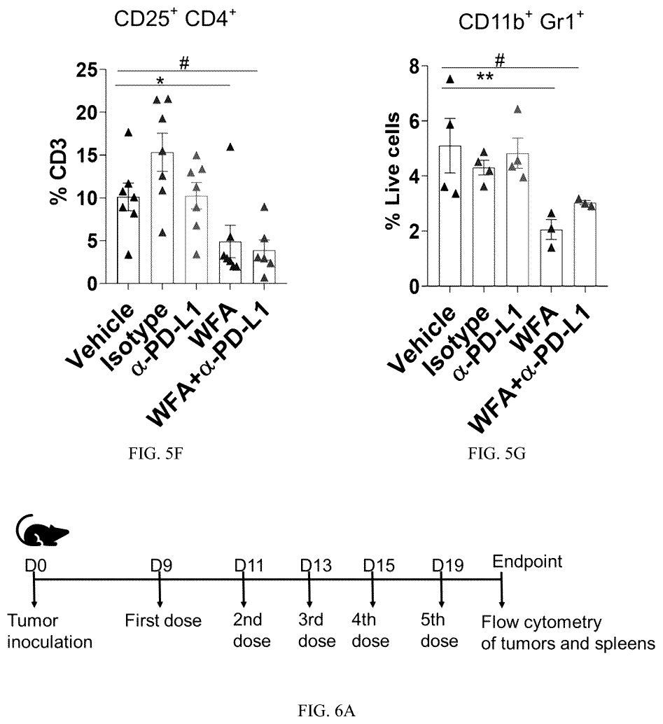

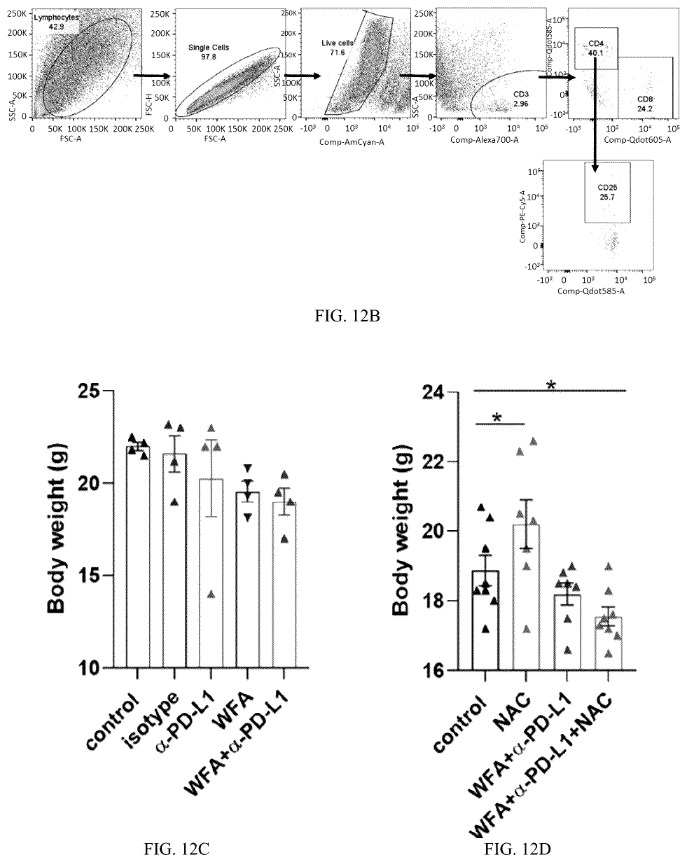

Since our in vitro data showed that WFA could sensitize NSCLC cells to α-PD-L1, we tested the effectiveness of a WFA and α-PD-L1 combination therapy in a LLC syngeneic mouse model. C57BL/6 mice were injected with 5×10⁵ LLC cells per flank and the treatment started eight days after tumor initiation (when the tumors were first palpable). Mice were randomized into vehicle control (10% DMSO, 90% Glyceryl Tri octanoate), isotype control (200 μg/mouse dose), WFA (5 mg/kg), α-PD-L1 (200 μg/mouse dose), or WFA+α-PD-L1 combination and treated every other day with a total of five treatments (FIG. 5A). While α-PD-L1 did not change the tumor size, WFA showed a non-significant reduction in tumor size (FIG. 5B). Although the individual treatments did not change tumor size, the combination treatment significantly reduced tumor growth (FIG. 5B). Moreover, we measured the body weight of the mice as an indicator of toxicity/disease burden. Interestingly, we found that the combination treatment did not significantly alter the body weight from the control mice (FIGS. 5B and 12C). At the endpoint, mice were euthanized for spleen and tumor collection and dissociation. To investigate the mechanism by which WFA increased the effectiveness of α-PD-L1 in vivo, we analyzed the changes in tumor-immune infiltration induced by different treatments, including both effector cell and immunosuppressive cell populations (FIGS. 12A-12B). Similar to our in vitro findings, WFA increased the levels of ecto-CRT in vivo compared to the control mice (FIG. 5C). Moreover, Flow cytometry analysis shows that WFA treatment skewed the TME towards a more anti-tumor phenotype by increasing CD8 T-cell infiltration (FIG. 5D-5E). However, only the combination treatment was observed to significantly increase the expression of T-cell activation markers CD69 and 41BB compared to vehicle control (FIG. 5E). Additionally, WFA targets immunosuppressive cell populations including CD11b+Gr1+MDSCs and CD25+CD4+T-regs, reducing their presence in the tumor (FIGS. 5F-5G). In summary, our in vivo results show that WFA sensitizes LLC tumors to α-PD-L1 and elicits an anti-tumor immune response by increasing CTL infiltration and targeting immunosuppressive cells.

### ROS Plays a Role in Inducing the Effectiveness of WFA+α-PD-L1 Combination

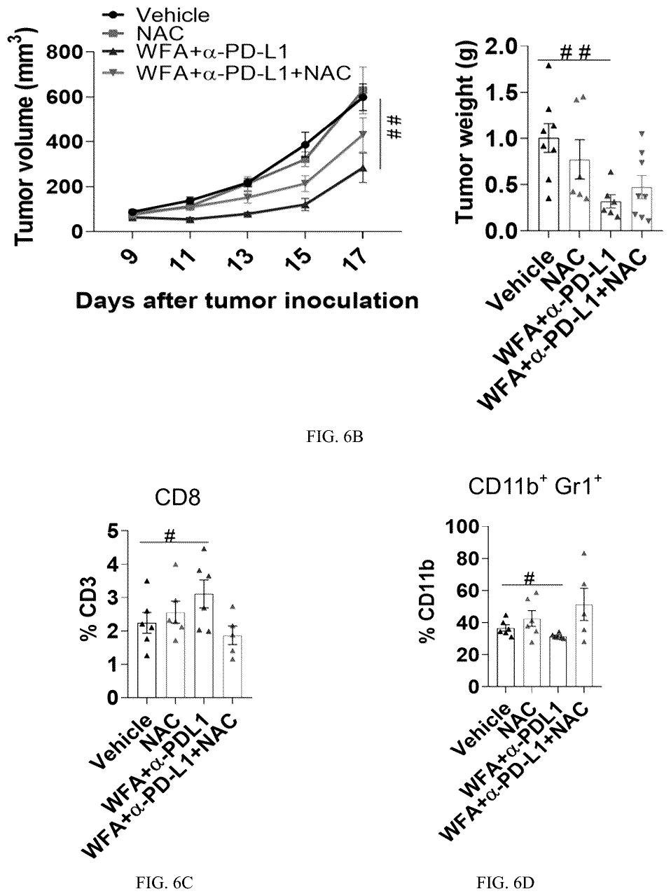

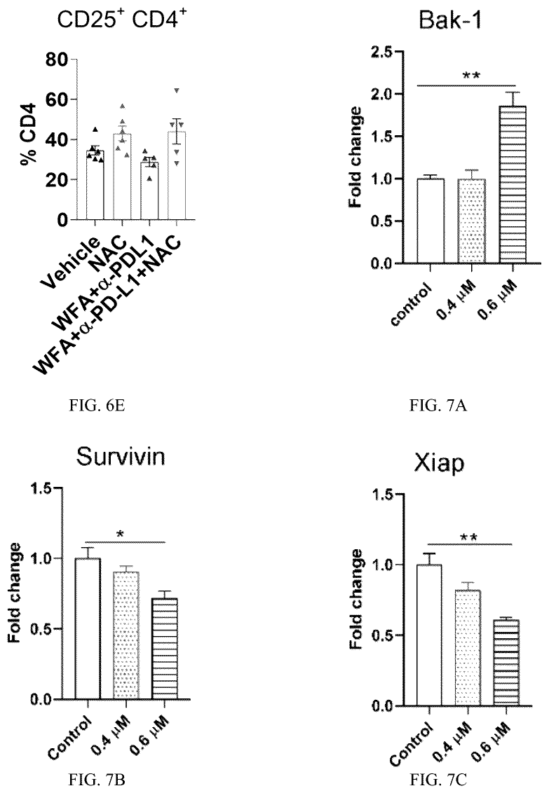

Since ROS production was responsible for inducing PD-L1 expression in vitro, we wanted to confirm whether WFA-mediated ROS production plays a role in tumor immunomodulation in vivo. To confirm the role of ROS, we treated LLC syngeneic mice with WFA+α-PD-L1 or WFA+α-PD-L1+NAC (100 mg/kg) (FIG. 6A). Similar to our previous findings, we found that the WFA+anti-PD-L1 combination significantly reduced tumor growth (FIG. 6B). However, the mice that received the added NAC treatment showed reduced effectiveness of the combination treatment. However, the change was not statistically significant from the WFA+α-PD-L1 combination treatment. At the endpoint, tumors and spleens were collected to measure the effect of NAC on tumor immune infiltration. Interestingly, we found that NAC treatment reduced CD8 T-cell infiltration (FIG. 6C) and increased the immunosuppressive cell populations when added to the WFA+α-PD-L1 combination (FIG. 6D-6E). In summary, although NAC did not completely reverse the effectiveness of the WFA+α-PD-L1 treatment, its partial rescue of the phenotype demonstrates that ROS plays a role in WFA mediated anti-tumor immunomodulation in vivo.

### DISCUSSION

A major finding of our study is that WFA induces ICD, increases the release of DAMPs, and increases PD-L1 expression in NSCLC, in addition to colorectal and breast cancer cells. We found that WFA-induced ICD in both murine and human NSCLC cell lines was characterized by the increased surface expression of CRT and the release of HMBGB-1. These molecules act as a ‘find me’ signal, attracting APC to the tumor site, and an ‘eat me’ signal that promotes DC activation and engulfment of tumor antigens [41]. Consequently, activated APC can present tumor antigens to prime an anti-tumor T-cell response [43,44]. We confirmed that WFA-induced DAMP release from treated LLC murine cell line leads to the increased activation of APC represented by syngeneic bone marrow-derived DC.

Another major finding of our study is the role of ROS in inducing PD-L1 expression in response to WFA in different types of cancer cell lines. Other studies showed that WFA causes cancer cell apoptosis by inducing mitochondrial dysfunction by inhibiting complex III of the electron transport chain, which in turn increases the intracellular levels of ROS [31]. Interestingly, this increase is not observed in non-cancerous cells, providing a potential explanation of WFA's selective toxicity [45,46]. We tested whether ROS plays a role in WFA-mediated PD-L1 increase by treating the cells with WFA and a ROS scavenger (NAC or GSH). Notably, we found that either NAC or GSH was able to completely reverse PD-L1 upregulation. Additionally, ROS scavengers inhibited WFA-induced increase in ecto-CRT. These findings prove that ROS plays a key role in WFA-mediated immunomodulation including ICD and PD-L1 upregulation. The literature shows that ROS can play either an immunostimulatory or an immunoinhibitory effect in the TME based on the type of cells from which it is produced and their specific location within the TME [47,48]. While ROS can induce ICD, increase tumor antigenicity, and reprogram tumor-associated macrophages, it also represents a major immunosuppressive mechanism when released by MDSCs or T-regs [49-53]. However, WFA was found to inhibit MDSCs production of ROS in a 4T1 mouse model suggesting that WFA plays a dual role in ROS signaling based on the cellular context [36].

Although the relationship between ROS and PD-L1 is complex, a few studies show that ROS usually induces PD-L1 upregulation by activating certain downstream transcription factors such as YAP-1, HIF-la, and NF-xB [51,54,55]. However, there are ROS-inducing drugs that reduce PD-L1 expression and vice versa [56,57]. We attempted to investigate the downstream regulator responsible for ROS-mediated PD-L1 upregulation using the IPA. By applying the mRNA expression fold change values from a publicly available dataset of WFA-treated MCF-7 breast cancer cells to a signaling network including ROS-PD-L1 connections, we found that both ROS and PD-L1 are predicted to be increased by WFA treatment. Additionally, IPA predictions show that multiple downstream regulators might be involved in ROS-mediated PD-L1 upregulation including STAT-3, YAP-1, and HSF-1 (FIG. 11). While testing all potential pathways is beyond the scope of this study, we pragmatically selected a couple to examine the ROS-mediated-PD-L1 upregulation. First, the combination of WFA with the pharmacologic inhibitors of STAT-3 or HIFla did not alter PD-L1 upregulation, suggesting that the pathways involving these transcription factors are not involved. In contrast, the NRF-2 inhibitor brusatol reversed PD-L1 upregulation, suggesting its role in WFA-induced PD-L1 upregulation, which is consistent with another study [58]. However, siRNA for NRF-2 did not rescue PD-L1 upregulation, questioning the role of NRF-2 in this process. Given the prediction of multiplicity of pathways involved in ROS-mediated PD-L1 upregulation, we infer that WFA-induced PD-L1 upregulation may involve more than a single pathway.

As our in vitro results show that WFA promotes ICD and PD-L1 upregulation, we tested the effectiveness of WFA+α-PD-L1 combination therapy in an in vivo LLC immunocompetent model. As others have shown, α-PD-L1 did not reduce tumor growth [59,60]. Although WFA-treated mice showed a reduction in tumor growth, the change was nonsignificant. The combination treatment of WFA+α-PD-L1 significantly reduced tumor growth. Moreover, flow cytometry analysis of tumor immune infiltration shows that WFA increased T-cell infiltration and reduced the immunosuppressive cell populations. We found that WFA increased CD8 T-cell infiltration and appeared to increase activation, however, the change in T-cell activation was not significant. The combination treatment showed a significant increase in CD8 T-cell percentages and activation markers CD69 and 4-1BB. We also found that both WFA and combination treatments reduced the levels of immunosuppressive MDSCs and T-regs. Overall, WFA can change a cold TME into a hot TME, increasing the effectiveness of α-PD-L1 in NSCLC [17,61]. Due to the opposing effects of ROS on anti-tumor immunity, we further investigated the role of ROS in WFA-mediated immunomodulation in vivo. Treating the LLC mice with NAC partially reversed the tumor growth inhibition observed with the WFA+α-PD-L1 combination treatment. Our flow cytometry analysis shows that NAC also reversed the immune phenotype observed with the combination treatment by reducing CD8 T-cell and increasing MDSC and T-reg infiltration. However, the change of tumor growth was not statistically significant from the combination treatment, which indicates that ROS only plays a partial role in WFA in vivo effectiveness. That suggests the involvement of other pathways or factors in the TME that may not be connected to ROS in WFA-mediated anti-cancer effectiveness. Further studies are required to identify these pathways. Although we observed WFA-mediated immunomodulation was ubiquitous in different types of cancer cells, further in vivo studies are needed to confirm the effectiveness of WFA and α-PD-L1 combination therapy in colorectal and breast cancer. Additionally, further studies are needed to investigate the effect of WFA on additional IC molecules to identify other effective combinatorial approaches. The choice of proper combinatorial therapy allows for personalized therapy. We monitored body weights as an indicator of toxicity and found that the WFA+α-PD-L1 combination treatment did not change the body weight compared to the vehicle treated controls. This suggests that the combination of WFA+α-PD-L1 may be safe and tolerable, however, this needs to be tested clinically.

### CONCLUSIONS

To summarize, our results demonstrate that WFA induces ICD in NSCLC and colorectal cancer cell lines and increases the release of DAMPs. Moreover, we found that WFA treatment increases PD-L1 expression in NSCLC, colorectal, and breast cancer cell lines. We investigated the underlying mechanism and found that these changes were mediated by ROS and were reversed by ROS scavengers like NAC and GSH. Moreover, for the first time, we showed that WFA sensitizes NSCLC to α-PD-L1 in an in vivo mouse model without causing additional toxicity. Our results provide a new combinatorial approach that can improve patient response to ICBs by converting an immunologically cold to an immunologically hot TME and prompts testing its effectiveness in a clinical setting.

### REFERENCES

1. Siegel, R. L.; Miller, K. D.; Fuchs, H. E.; Jemal, A. Cancer statistics, 2022. CA A Cancer J. Clin. 2022, 72, 7-33.
2. Centers for Disease Control and Prevention. U.S. Cancer Statistics Lung Cancer Stat Bite; US Department of Health and Human Services: Washington, DC, USA, 2022.
3. He, X.; Xu, C. Immune checkpoint signaling and cancer immunotherapy. Cell Res. 2020, 30, 660-669.
4. Sanchez-Paulete, A. R.; Teijeira, A.; Cueto, F. J.; Garasa, S.; Perez-Gracia, J. L.; Sanchez-Arriez, A.; Sancho, D.; Melero, I. Antigen cross-presentation and T-cell cross-priming in cancer immunology and immunotherapy. Ann. Oncol. 2017, 28, xii44-xii55.
5. Chambers, C. A.; Allison, J. P. Costimulatory regulation of T cell function. Curr. Opin. Cell Biol. 1999, 11, 203-210.
6. Borghaei, H.; Paz-Ares, L.; Horn, L.; Spigel, D. R.; Steins, M.; Ready, N. E.; Chow, L. Q.; Vokes, E. E.; Felip, E.; Holgado, E.; et al. Nivolumab versus Docetaxel in Advanced Nonsquamous Non-Small-Cell Lung Cancer. N. Engl. J. Med. 2015, 373, 1627-1639.
7. Principe, N.; Kidman, J.; Goh, S.; Tilsed, C. M.; Fisher, S. A.; Fear, V. S.; Forbes, C. A.; Zemek, R. M.; Chopra, A.; Watson, M.; et al. Tumor Infiltrating Effector Memory Antigen-Specific CD8(+) T Cells Predict Response to Immune Checkpoint Therapy. Front. Immunol. 2020, 11, 584423.
8. Tang, S.; Qin, C.; Hu, H.; Liu, T.; He, Y; Guo, H.; Yan, H.; Zhang, J.; Tang, S.; Zhou, H. Immune Checkpoint Inhibitors in Non-Small Cell Lung Cancer: Progress, Challenges, and Prospects. Cells 2022, 11, 320.
9. Wellenstein, M. D.; de Visser, K. E. Cancer-Cell-Intrinsic Mechanisms Shaping the Tumor Immune Landscape. Immunity 2018, 48, 399-416.
10. Bai, R.; Chen, N.; Li, L.; Du, N.; Bai, L.; Lv, Z.; Tian, H.; Cui, J. Mechanisms of Cancer Resistance to Immunotherapy. Front. Oncol. 2020, 10, 1290.
11. Bai, R.; Lv, Z.; Xu, D.; Cui, J. Predictive biomarkers for cancer immunotherapy with immune checkpoint inhibitors. Biomark. Res. 2020, 8, 34.
12. Kowanetz, M.; Zou, W.; Gettinger Scott, N.; Koeppen, H.; Kockx, M.; Schmid, P.; Kadel Edward, E.; Wistuba, I.; Chaft, J.; Rizvi Naiyer, A.; et al. Differential regulation of PD-L1 expression by immune and tumor cells in NSCLC and the response to treatment with atezolizumab (anti-PD-L1). Proc. Natl. Acad. Sci. USA 2018, 115, E10119-E10126.
13. Wang, L.; Hu, Y; Wang, S.; Shen, J.; Wang, X. Biomarkers of immunotherapy in non-small cell lung cancer (Review). Oncol. Lett. 2020, 20, 139.
14. Sahin, I. H.; Akce, M.; Alese, O.; Shaib, W.; Lesinski, G. B.; E1-Rayes, B.;Wu, C. Immune checkpoint inhibitors for the treatment of MSI-H/MMR-D colorectal cancer and a perspective on resistance mechanisms. Br. J. Cancer 2019, 121, 809-818.
15. Spaas, M.; Lievens, Y Is the Combination of Immunotherapy and Radiotherapy in Non-small Cell Lung Cancer a Feasible and Effective Approach? Front. Med. 2019, 6, 244.
16. Shao, X.; Wang, X.; Guo, X.; Jiang, K.; Ye, T.; Chen, J.; Fang, J.; Gu, L.; Wang, S.; Zhang, G.; et al. STAT3 Contributes To Oncolytic Newcastle Disease Virus-Induced Immunogenic Cell Death in Melanoma Cells. Front. Oncol. 2019, 9, 436.
17. Liu, YT.; Sun, Z. J. Turning cold tumors into hot tumors by improving T-cell infiltration. Theranostics 2021, 11, 5365-5386.
18. Luo, M.; Wang, F.; Zhang, H.; To, K. K. W.; Wu, S.; Chen, Z.; Liang, S.; Fu, L. Mitomycin C enhanced the efficacy of PD-L1 blockade in non-small cell lung cancer. Signal Transduct. Target. Ther. 2020, 5, 141.
19. Fournel, L.; Wu, Z.; Stadler, N.; Damotte, D.; Lococo, F.; Boulle, G.; Segal-Bendirdjian, E.; Bobbio, A.; Icard, P.; Tredaniel, J.; et al. Cisplatin increases PD-L1 expression and optimizes immune check-point blockade in non-small cell lung cancer. Cancer Lett. 2019, 464, 5-14.
20. Florescu, M.; Cinteza, M.; Vinereanu, D. Chemotherapy-induced Cardiotoxicity. Maedica 2013, 8, 59-67.
21. Li, C.; Bhatti, S. A.; Ying, J. Immune Checkpoint Inhibitors-Associated Cardiotoxicity.

### Cancers 2022, 14, 1145.

22. Dutta, R.; Khalil, R.; Green, R.; Mohapatra, S. S.; Mohapatra, S. Withania Somnifera (Ashwagandha) and Withaferin A: Potential in Integrative Oncology. Int. J. Mol. Sci. 2019, 20, 5310.
23. Kakar, S. S.; Parte, S.; Carter, K.; Joshua, I. G.; Worth, C.; Rameshwar, P.; Ratajczak, M. Z. Withaferin A (WFA) inhibits tumor growth and metastasis by targeting ovarian cancer stem cells. Oncotarget 2017, 8, 74494-74505.
24. Kyakulaga, A. H.; Aqil, F.; Munagala, R.; Gupta, R. C. Withaferin A inhibits Epithelial to Mesenchymal Transition in Non-Small Cell Lung Cancer Cells. Sci. Rep. 2018, 8, 15737.
25. Ting, L.-L.; Chou, A. S.-B.; Hsieh, C.-H.; Hsiung, S.-C.; Pang, S.-T.; Liao, S.-K. Withaferin A targeting both cancer stem cells and metastatic cancer stem cells in the UP-LN1 carcinoma cell model. J. Cancer Metastasis Treat. 2016, 2, 29-40.
26. Shiragannavar, V. D.; Gowda, N. G. S.; Kumar, D. P.; Mirshahi, F.; Santhekadur, P. K. Withaferin A Acts as a Novel Regulator of Liver X Receptor-α in HCC. Front. Oncol. 2020, 10, 628506.
27. Heyninck, K.; Lahtela-Kakkonen, M.; Van der Veken, P.; Haegeman, G.; Vanden Berghe, W. Withaferin A inhibits NF-kappaB activation by targeting cysteine 179 in IKKO. Biochem. Pharm. 2014, 91, 501-509.
28. Lee, I. C.; Choi, B. Y Withaferin-A-A Natural Anticancer Agent with Pleitropic Mechanisms of Action. Int. J. Mol. Sci. 2016, 17, 290.
29. Kunimasa, K.; Nagano, T.; Shimono, Y; Dokuni, R.; Kiriu, T.; Tokunaga, S.; Tamura, D.; Yamamoto, M.; Tachihara, M.; Kobayashi, K.; et al. Glucose metabolism-targeted therapy and withaferin A are effective for epidermal growth factor receptor tyrosine kinase inhibitor-induced drug-tolerant persisters. Cancer Sci. 2017, 108, 1368-1377.
30. Ghosh, K.; De, S.; Das, S.; Mukherjee, S.; Sengupta Bandyopadhyay, S. Withaferin A Induces ROS-Mediated Paraptosis in Human Breast Cancer Cell-Lines MCF-7 and MDA-MB-231. PLoS ONE 2016, 11, e0168488.
31. Sehrawat, A.; Samanta, S. K.; Hahm, E. R.; St Croix, C.; Watkins, S.; Singh, S. V. Withaferin A-mediated apoptosis in breast cancer cells is associated with alterations in mitochondrial dynamics. Mitochondrion 2019, 47, 282-293.
32. Guo, R.; Gan, L.; Lau, W. B.; Yan, Z.; Xie, D.; Gao, E.; Christopher, T. A.; Lopez, B. L.; Ma, X.; Wang, Y Withaferin A Prevents Myocardial Ischemia/Reperfusion Injury by Upregulating AMP-Activated Protein Kinase-Dependent B-Cell Lymphoma2 Signaling. Circ. J. 2019, 83, 1726-1736.
33. Liu, X.; Chen, L.; Liang, T.; Tian, X. D.; Liu, Y; Zhang, T. Withaferin A induces mitochondrial-dependent apoptosis in non-small cell lung cancer cells via generation of reactive oxygen species. J. BUON 2017, 22, 244-250.
34. Malik, F.; Singh, J.; Khajuria, A.; Suri, K. A.; Satti, N. K.; Singh, S.; Kaul, M. K.; Kumar, A.; Bhatia, A.; Qazi, G. N. A standardized root extract of Withania somnifera and its major constituent withanolide-A elicit humoral and cell-mediated immune responses by up regulation of Thl-dominant polarization in BALB/c mice. Life Sci. 2007, 80, 1525-1538.
35. Tharakan, A.; Shukla, H.; Benny, I. R.; Tharakan, M.; George, L.; Koshy, S. Immunomodulatory Effect of Withania somnifera (Ashwagandha) Extract-A Randomized, Double-Blind, Placebo Controlled Trial with an Open Label Extension on Healthy Participants. J. Clin. Med. 2021, 10, 3644.
36. Sinha, P.; Ostrand-Rosenberg, S. Myeloid-derived suppressor cell function is reduced by Withaferin A, a potent and abundant component of Withania somnifera root extract. Cancer Immunol. Immunother. 2013, 62, 1663-1673.
37. Hossain, D. M. S.; Javaid, S.; Cai, M.; Zhang, C.; Sawant, A.; Hinton, M.; Sathe, M.; Grein, J.; Blumenschein, W.; Pinheiro, E. M.; et al. Dinaciclib induces immunogenic cell death and enhances anti-PDl-mediated tumor suppression. J. Clin. Investig. 2018, 128, 644-654.
38. Kepp, O.; Menger, L.; Vacchelli, E.; Locher, C.; Adjemian, S.; Yamazaki, T.; Martins, I.; Sukkurwala, A. Q.; Michaud, M.; Senovilla, L.; et al. Crosstalk between ER stress and immunogenic cell death. Cytokine Growth Factor Rev. 2013, 24, 311-318.
39. Fucikova, J.; Kepp, O.; Kasikova, L.; Petroni, G.; Yamazaki, T.; Liu, P.; Zhao, L.; Spisek, R.; Kroemer, G.; Galluzzi, L. Detection of immunogenic cell death and its relevance for cancer therapy. Cell Death Dis. 2020, 11, 1013.
40. Nath, S.; Obaid, G.; Hasan, T. The Course of Immune Stimulation by Photodynamic Therapy: Bridging Fundamentals of Photochemically Induced Immunogenic Cell Death to the Enrichment of T-Cell Repertoire. Photochem. Photobiol. 2019, 95, 1288-1305.
41. Galluzzi, L.; Kepp, O.; Hett, E.; Kroemer, G.; Marincola, F. M. Immunogenic cell death in cancer: Concept and therapeutic implications. J. Transl. Med. 2023, 21, 162.
42. Xia, S.; Miao, Y; Liu, S. Withaferin Ainduces apoptosis by ROS-dependent mitochondrial dysfunction in human colorectal cancer cells. Biochem. Biophys. Res. Commun. 2018, 503, 2363-2369.
43. Krysko, D. V; Garg, A. D.; Kaczmarek, A.; Krysko, O.; Agostinis, P.; Vandenabeele, P. Immunogenic cell death and DAMPs incancer therapy. Nat. Rev. Cancer 2012, 12, 860-875.
44. Di Blasio, S.; Wortel, I. M.; van Bladel, D. A.; de Vries, L. E.; Duiveman-de Boer, T.; Worah, K.; de Haas, N.; Buschow, S. I.; de Vries, I. J.; Figdor, C. G.; et al. Human CDlc+DCs are critical cellular mediators of immune responses induced by immunogenic cell death. Oncoimmunology 2016, 5, e1192739.
45. Batumalaie, K.; Amin, M. A.; Murugan, D. D.; Sattar, M. Z. A.; Abdullah, N. A. Withaferin A protects against palmitic acid-induced endothelial insulin resistance and dysfunction through suppression of oxidative stress and inflammation. Sci. Rep. 2016, 6, 27236.
46. Zhou, Z.; Xiang, W.; Jiang, Y; Tian, N.; Wei, Z.; Wen, X.; Wang, W.; Liao, W.; Xia, X.; Li, Q.; et al. Withaferin A alleviates traumatic brain injury induced secondary brain injury via suppressing apoptosis in endothelia cells and modulating activation in the microglia. Eur. J. Pharmacol. 2020, 874, 172988.
47. Bailly, C. Regulation of PD-L1 expression on cancer cells with ROS-modulating drugs. Life Sci. 2020, 246, 117403.
48. Choi, E. J.; Jeon, C. H.; Lee, I.-K. Ferric Ammonium Citrate Upregulates PD-L1 Expression through Generation of Reactive Oxygen Species. J. Immunol. Res. 2022, 2022, 6284124.
49. Roux, C.; Jafari, S. M.; Shinde, R.; Duncan, G.; Cescon, D. W.; Silvester, J.; Chu, M. F.; Hodgson, K.; Berger, T.; Wakeham, A.; et al. Reactive oxygen species modulate macrophage immunosuppressive phenotype through the up-regulation of PD-L1. Proc. Natl. Acad. Sci. USA 2019, 116, 4326-4335.
50. Xue, J.; Li, L.; Li, N.; Li, F.; Qin, X.; Li, T.; Liu, M. Metformin suppresses cancer cell growth in endometrial carcinoma by inhibiting PD-L1. Eur. J. Pharmacol. 2019, 859, 172541.
51. Zhou, X.; Wang, W.; Wang, C.; Zheng, C.; Xu, X.; Ni, X.; Hu, S.; Cai, B.; Sun, L.; Shi, K.; et al. DPP4 Inhibitor Attenuates Severe Acute Pancreatitis-Associated Intestinal Inflammation via Nrf2 Signaling. Oxidative Med. Cell. Longev. 2019, 2019, 6181754.
52. Zhu, S.; Liu, S.; Wang, L.; Ding, W.; Sha, J.; Qian, H.; Lu, Y Brusatol Protects HepG2 Cells against Oxygen-Glucose Deprivation-Induced Injury via Inhibiting Mitochondrial Reactive Oxygen Species-Induced Oxidative Stress. Pharmacology 2019, 105, 416-423.
53. Kotsafti, A.; MScarpa; Castagliuolo, I.; Scarpa, M. Reactive Oxygen Species and Antitumor Immunity—From Surveillance to Evasion. Cancers 2020, 12, 1748.
54. Wang, L.; Kuang, Z.; Zhang, D.; Gao, Y; Ying, M.; Wang, T. Reactive oxygen species in immune cells: Anew antitumor target. Biomed. Pharmacother. 2021, 133, 110978.
55. Hassannia, B.; Wiernicki, B.; Ingold, I.; Qu, F.; Van Herck, S.; Tyurina, YY; Bayir, H.; Abhari, B. A.; Angeli, J. P. F.; Choi, S. M.; et al. Nano-targeted induction of dual ferroptotic mechanisms eradicates high-risk neuroblastoma. J. Clin. Investig. 2018, 128, 3341-3355.
56. Ohl, K.; Tenbrock, K. Reactive Oxygen Species as Regulators of MDSC-Mediated Immune Suppression. Front. Immunol. 2018, 9, 2499.
57. Shi, C.; Liu, T.; Guo, Z.; Zhuang, R.; Zhang, X.; Chen, X. Reprogramming Tumor-Associated Macrophages by Nanoparticle-Based Reactive Oxygen Species Photogeneration. Nano Lett. 2018, 18, 7330-7342.
58. Zhu, B.; Tang, L.; Chen, S.; Yin, C.; Peng, S.; Li, X.; Liu, T.; Liu, W.; Han, C.; Stawski, L.; et al. Targeting the upstream transcriptional activator of PD-L1 as an alternative strategy in melanoma therapy. Oncogene 2018, 37, 4941-4954.
59. Luo, F.; Luo, M.; Rong, Q.-X.; Zhang, H.; Chen, Z.; Wang, F.; Zhao, H.-Y; Fu, L.-W. Niclosamide, an antihelmintic drug, enhances efficacy of PD-1/PD-L1 immune checkpoint blockade in non-small cell lung cancer. J. Immunother. Cancer 2019, 7, 245.
60. Veronica Olivo, P.; Damienne, M.; Alexander, M. A. v. d. W.; Natasja, G. L.; Rianne, B.; Relinde, IYL.; Dario, N.; Jan, T.; Ala, Y; Ludwig, J. D.; et al. Releasing the brakes of tumor immunity with anti-PD-L1 and pushing its accelerator with L19-IL2 cures poorly immunogenic tumors when combined with radiotherapy. J. Immunother. Cancer 2021, 9, e001764.
61. Duan, Q.; Zhang, H.; Zheng, J.; Zhang, L. Turning Cold into Hot: Firing up the Tumor Microenvironment. Trends Cancer 2020, 6, 605-618.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A method of treating a cancer in a subject in need thereof comprising administering to the subject a therapeutically effective amount of withaferin A and an immune checkpoint inhibitor.

2. The method of claim 1, wherein the cancer is resistant or has developed resistance to treatment with the immune checkpoint blocker alone.

3. The method of claim 1, wherein the cancer is resistant or has developed resistance to treatment with the immune checkpoint blocker in combination with another therapeutic agent other than withaferin A.

4. The method of claim 1, wherein the cancer is susceptible to developing resistance to treatment with the immune checkpoint blocker alone.

5. The method of claim 1, wherein the cancer is susceptible to developing resistance to treatment with the immune checkpoint blocker in combination with another therapeutic agent other than withaferin A.

6. The method of claim 1, wherein the cancer is selected from basal cell carcinoma, bladder cancer, breast cancer, cervical cancer, cholangiocarcinoma, colorectal cancer, endometrial cancer, esophageal cancer, head and neck cancer, hepatocellular carcinoma, Hodgkin lymphoma, melanoma, mesothelioma, Merkel cell carcinoma, non-small cell lung cancer, renal cell carcinoma, small cell lung cancer, squamous cell carcinoma, stomach cancer, and urothelial carcinoma.

7. The method of claim 1, wherein the cancer is selected from breast cancer, colorectal cancer, and non-small cell lung cancer.

8. The method of claim 1, wherein the immune checkpoint blocker comprises an inhibitor of CTLA-4.

9. The method of claim 1, wherein the immune checkpoint blocker comprises an inhibitor of CTLA-4 selected from ipilimumab and tremelimumab.

10. The method of claim 1, wherein the immune checkpoint blocker comprises an inhibitor of PD-1.

11. The method of claim 1, wherein the immune checkpoint blocker comprises an inhibitor of PD-1 selected from pembrolizumab, nivolumab, cemiplimab, dostarlimab, retafanlimab, toripalimab, vopratelimab (JTX-4014), spartalizumab (PDR001), cambrelizumab (SHR1210), sintilimab (IBI308), tislelizumab (BGB-A317), INCMGA00012 (MGA012), AMP-224, AMP-514 (MEDI0680), and acrixolimab (YBK-006).

12. The method of claim 1, wherein the immune checkpoint blocker comprises an inhibitor of PD-1 selected from nivolumab, pembrolizumab, cemiplimab, and dostarlimab.

13. The method of claim 1, wherein the immune checkpoint blocker comprises an inhibitor of PD-Li.

14. The method of claim 1, wherein the immune checkpoint blocker comprises an inhibitor of PD-Li selected from atezolizumab, avelumab, durvalumab, KN035, cosibelimab (CK-301), AUNP12, CA-170, and BMS-986189.

15. The method of claim 1, wherein the immune checkpoint blocker comprises an inhibitor of PD-Li selected from atezolizumab, avelumab, and durvalumab.

16. The method of claim 1, further comprising administering one or more additional therapeutic agents.

17. The method of claim 1, further comprising administering a therapeutically effective amount of ionizing radiation.

18. The method of claim 1, wherein the subject is a human.
